SEQUENCE LISTING (6 sequences)

SEQ ID NO: 1 - DNA, 470 bp, synthetic construct
  source 1..470: mol_type = other DNA
ctgacgtcaccctctaggcgtctggataggacgatcctggctactcccattcagggctgctgtccagtgctgctttattggcagtgctgccagggtctccgttagctctctgcaaattgccttcctttctgctcctcctactccctccttcccccatagaatttttcttttcattgcccactttactgttttggctccagactgtcgttaagaatgtacagcctaattctggtgtgtttcgggatattcttctgtccagtattctggaagggcggggaggcatggcagcgttttacttgacgttgatggtgctgtgaagtccattctttcctctgcaagactactgactatgcagaaatttatcgaagcggattattatgaactagactggtattatgaagaatgctcggatggtaattatggcccctgcaaaacagagccgggatgtataggggtattgtctccttctg

SEQ ID NO: 2 - DNA, 470 bp, synthetic construct
  source 1..470: mol_type = other DNA
ttgacgttattttttaggcgtttggataggacgattttggttatttttatttagggttgttgtttagtgttgttttattggtagtgttgttagggttttcgttagttttttgtaaattgtttttttttttgttttttttatttttttttttttttatagaatttttttttttattgtttattttattgttttggttttagattgtcgttaagaatgtatagtttaattttggtgtgtttcgggatatttttttgtttagtattttggaagggcggggaggtatggtagcgttttatttgacgttgatggtgttgtgaagtttatttttttttttgtaagattattgattatgtagaaatttatcgaagcggattattatgaattagattggtattatgaagaatgttcggatggtaattatggtttttgtaaaatagagtcgggatgtataggggtattgtttttttttg

SEQ ID NO: 3 - DNA, 25 bp, synthetic construct
  source 1..25: mol_type = other DNA
ttttattgttttggttttagattgt

SEQ ID NO: 4 - DNA, 25 bp, synthetic construct
  source 1..25: mol_type = other DNA
gttgatggtgttgtgaagtttattt

SEQ ID NO: 5 - DNA, 25 bp, synthetic construct
  source 1..25: mol_type = other DNA
aaataaacttcacaacaccatcaac

SEQ ID NO: 6 - DNA, 25 bp, synthetic construct
  source 1..25: mol_type = other DNA
acaatctaaaaccaaaacaataaaa

(19) United States
(12) Patent Application Publication
(10) Pub. No.: US 2025/0223645 A1
(43) Pub. Date: Jul. 10, 2025

(54) NEURONAL METHYLATION SIGNATURES FROM CELL FREE DNA AS A PRE-SYMPTOMATIC NEURODEGENERATIVE CONDITION SURVEILLANCE DIAGNOSTIC

(71) Applicant: RESONANT LLC, Heber City, UT (US)

(72) Inventors: Chad POLLARD, Heber City, UT (US); Timothy JENKINS, Heber City, UT (US)

(21) Appl. No.: 18/847,141

(22) PCT Filed: Mar. 14, 2023
(86) PCT No.: PCT/US2023/064344

§ 371 Date: Sep. 13, 2024

Related U.S. Application Data
(60) Provisional application No. 63/488,268, filed on Mar. 3, 2023.
(60) Provisional application No. 63/319,916, filed on Mar. 15, 2022.

(51) Int. Cl. C12Q 1/6883; G16H 50/30

(52) U.S. Cl. CPC C12Q 1/6883; G16H 50/30; C12Q 2600/106; C12Q 2600/154; C12Q 2600/158

(57) ABSTRACT

Disclosed herein is a blood based two-part diagnostic tool designed to diagnose pre-symptomatic neurodegenerative disease. Part one consists of an assay designed to amplify and sequence pre-specified regions of cell free DNA. Part two consists of a python-derived pipeline that analyzes methylation signatures to identify neuronal-derived DNA and provide a diagnosis of pre-symptomatic neurodegenerative disease.

### CROSS-REFERENCE TO RELATED APPLICATIONS

This application is an International Application which claims priority to U.S. Provisional Application Nos. 63/319,916 filed on Mar. 15, 2022, and 63/488,268 filed on Mar. 3, 2023, each of which are incorporated herein by reference in their entireties and for all purposes.

### SEQUENCE LISTING

The material in the accompanying Sequence Listing is hereby incorporated by reference in its entirety. The accompanying file, named “062097-501001WO_SL_ST26.xml” was created on Mar. 14, 2023 and is 7,113 bytes.

### FIELD

Disclosed herein is a blood based two-part diagnostic tool designed to diagnose a pre-symptomatic neurodegenerative condition. Part one consists of an assay designed to amplify and sequence pre-specified regions of cell free DNA. Part two consists of a python-derived pipeline that analyzes methylation signatures to identify neuronal-derived DNA and provide a diagnosis of pre-symptomatic neurodegenerative conditions.

### BACKGROUND

The current standard of care for the diagnosis of neurodegenerative conditions, such as Alzheimer's and Parkinson's disease, uses a series of memory and physical tests to identify any onset of symptoms associated with the condition. If associated symptoms are identified, this indicates that neuron cell death has already progressed beyond repair. There is currently no known method for diagnosing neurodegenerative condition prior to onset of symptoms.

There have been significant therapeutic breakthroughs in research whose results show promising evidence of treatment that slows neuron cell death. Yet, without a presymptomatic diagnosis, treatment cannot be administered prior to irreparable neuronal cell death.

The present disclosure addresses these and other concerns in the art.

### SUMMARY

The instant technology generally relates to a pre-screening method that can identify neurodegenerative condition prior to onset of symptoms. The disclosed methods could be used to diagnose pre-symptomatic neurodegenerative disease and paired with the proper therapeutic to prevent or delay the onset of disease in the patient.

In one aspect, the present disclosure provides a method of treatment of a subject, wherein the subject has or is at risk of having a neurodegenerative condition, the method including: (i) obtaining cell-free DNA from a blood sample from the subject; (ii) analyzing a methylation pattern of the cell-free DNA; (iii) determining a percentage of cell-free DNA from neurons; (iv) comparing the percentage of cell-free DNA from neurons to a control; and (v) administering a therapeutic agent that treats or prevents the neurodegenerative condition when the percentage of cell-free DNA from neurons is greater than the control.

In some embodiments, step (ii) includes analyzing a methylation pattern of whole amplicons of DNA. In some embodiments, the whole amplicons are at least about 50 base pairs (bp) in length. In some embodiments, the whole amplicons are between about 50 and about 500 base pairs in length.

In some embodiments, the therapeutic agent is administered when the percentage of cell-free DNA from neurons is greater than about 5%. In some embodiments, the therapeutic agent is administered when the percentage of cell-free DNA from neurons is greater than about 7%. In some embodiments, the therapeutic agent is administered when the percentage of cell-free DNA from neurons is greater than about 9%.

In another aspect, the present disclosure provides a method of treatment of a subject, wherein the subject has or is at risk of having a neurodegenerative condition, the method including: (i) obtaining cell-free DNA from a blood sample from the subject; (ii) analyzing a methylation pattern of the cell-free DNA; (iii) determining a percentage of cell-free DNA from neurons; and (iv) administering a therapeutic agent that treats or prevents the neurodegenerative condition when the percentage of cell-free DNA from neurons is greater than about 5%.

In another aspect, the present disclosure provides a method of analyzing a biological sample of a subject, the method including: (i) obtaining cell-free DNA from a blood sample from the subject; (ii) analyzing a methylation pattern of the cell-free DNA; (iii) determining a percentage of cell-free DNA from neurons; and (iv) comparing the percentage of cell-free DNA from neurons to a control.

In another aspect, the present disclosure provides a method of measuring neuron cell death in a subject, the method including: (i) obtaining cell-free DNA from a blood sample from the subject; (ii) analyzing a methylation pattern of the cell-free DNA; (iii) determining a percentage of cell-free DNA from neurons; and (iv) comparing the percentage of cell-free DNA from neurons to a control.

In another aspect, the present disclosure provides a method of selecting a patient for treatment with a therapeutic agent for treatment of a neurodegenerative condition, the method including: (i) obtaining cell-free DNA from a blood sample from the subject; (ii) analyzing a methylation pattern of the cell-free DNA; (iii) determining a percentage of cell-free DNA from neurons; and (iv) comparing the percentage of cell-free DNA from neurons to a control; wherein the patient is selected for treatment when the percentage of cell-free DNA from neurons is greater than the control.

In some embodiments, step (ii) includes analyzing a methylation pattern of whole amplicons of DNA. In some embodiments, the whole amplicons are at least about 50 base pairs (bp) in length. In some embodiments, the whole amplicons are between about 50 and about 500 base pairs in length.

In some embodiments, the patient is selected for treatment when the percentage of cell-free DNA from neurons is greater than about 5%. In some embodiments, the patient is selected for treatment when the percentage of cell-free DNA from neurons is greater than about 7%. In some embodiments, the patient is selected for treatment when the percentage of cell-free DNA from neurons is greater than about 9%.

In some embodiments, a percentage of cell-free DNA from neurons greater than the control indicates an increased risk of neurodegenerative condition or traumatic brain injury.

In another aspect, the present disclosure provides a computer product including a non-transitory computer readable medium storing a plurality of instructions that when executed control a computer system to analyze a biological sample from a subject to determine the risk of neurodegenerative condition in the subject, the biological sample including cell-free DNA, the instructions including: (i) identifying a first DNA methylation pattern that occurs in a neuron at a rate above a threshold, wherein the first DNA methylation pattern includes methylation at one or more methylated regions and optionally includes no methylation at one or more unmethylated regions; (ii) analyzing a second DNA methylation pattern of the cell-free DNA; and (iii) computing a relative abundance of the one or more methylated regions and optionally the one or more unmethylated regions in the cell-free DNA; and (iv) determining the risk of neurodegenerative condition in the subject by comparing the relative abundance to a control.

In another aspect, the present disclosure provides a method for determining efficacy of a potential treatment of a neurodegenerative condition, the method including: (i) obtaining cell-free DNA from a blood sample from a plurality of subjects, wherein the subjects have been administered the potential treatment; (ii) analyzing a methylation pattern of the cell-free DNA; (iii) determining a percentage of cell-free DNA from neurons; and (iv) comparing the percentage of cell-free DNA from neurons to a control; wherein the potential treatment is efficacious when the percentage of cell-free DNA from neurons is less than the control.

In some embodiments, steps (i)-(iv) are repeated at least once. In some embodiments, steps (i)-(iv) are repeated weekly.

In some embodiments, determining a percentage of cell-free DNA from neurons includes comparing the methylation pattern of the cell-free DNA to a neuronal DNA methylation pattern, wherein the neuronal DNA methylation pattern includes methylation at one or more methylated regions and optionally comprises no methylation at one or more unmethylated regions.

In another aspect, the present disclosure provides a computer-implemented method of analyzing a biological sample, including: (i) identifying a first DNA methylation pattern that occurs in a neuron at a rate above a threshold, wherein the first DNA methylation pattern includes methylation at one or more methylated regions and optionally includes no methylation at one or more unmethylated regions; (ii) analyzing a second DNA methylation pattern of the cell-free DNA; and (iii) computing a relative abundance of the one or more methylated regions and optionally the one or more unmethylated regions in the cell-free DNA; and (iv) determining the risk of neurodegenerative disease in the subject by comparing the relative abundance to a control.

In some embodiments, step (ii) includes analyzing a methylation pattern of whole amplicons of DNA.

In some embodiments, the whole amplicons are at least about 50 base pairs (bp) in length. In some embodiments, the whole amplicons are between about 50 and about 500 base pairs in length.

In some embodiments, the control is a percentage of cell-free DNA from neurons in a blood sample from an untreated subject, a blood sample from the subjects prior to treatment, or a threshold. In some embodiments, the threshold is a percentage of cell-free DNA from neurons greater than about 5%. In some embodiments, the threshold is a percentage of cell-free DNA from neurons greater than about 7%. In some embodiments, the threshold is a percentage of cell-free DNA from neurons greater than about 9%.

In some embodiments, the neurodegenerative disease is selected from Alzheimer's disease, Huntington disease, Parkinson's disease, amyotrophic lateral sclerosis (ALS), ataxia, multiple sclerosis, multiple system atrophy, or concussion.

In some embodiments, the neuron is a motor neuron, a sensory neuron, an interneuron, a dopaminergic neuron, cholinergic neuron, a GABAergic neuron, a glutamatergic neuron, or a cortical neuron. In some embodiments, the neuron is from the forebrain, midbrain, or hindbrain. In some embodiments, the neuron is from the frontal lobe, temporal lobe, parietal lobe, occipital lobe, cerebellum, or brain stem.

In some embodiments, analyzing the methylation pattern includes converting 5-methylcytosine in the cell-free DNA to a different nucleotide. In some embodiments, converting includes bisulfite conversion or enzymatic conversion.

In some embodiments, the subject has mild cognitive impairment. In some embodiments, the subject is over 45 years of age. In some embodiments, including selecting a subject over 45 years of age. In some embodiments, the subject has no symptoms of the neurodegenerative condition. In some embodiments, including selecting a subject having mild cognitive impairment.

In another aspect, the present disclosure provides a method of treating a subject having a mild traumatic brain injury, including: (i) selecting a subject at risk for mild traumatic brain injury; (ii) obtaining cell-free DNA from a blood sample from the subject; (iii) analyzing a methylation pattern of the cell-free DNA; (iv) determining a percentage of cell-free DNA from neurons; and (v) comparing the percentage of cell-free DNA from neurons to a control; (vi) treating the subject for mild traumatic brain injury when the percentage of cell-free DNA from neurons is greater than the control.

In some embodiments, treating the subject includes administering a therapeutic agent that treats one or more symptoms of mild traumatic brain injury. In some embodiments, further including repeating steps (ii) to (v) at a timepoint after treatment. In some embodiments, treatment is discontinued when the percentage of cell-free DNA from neurons at the timepoint is less than or equal to the control. In some embodiments, treatment is continued when the percentage of cell-free DNA from neurons at the timepoint is greater than the control.

In another aspect, the present disclosure provides a kit including a first plurality of oligonucleotides, wherein each oligonucleotide in the first plurality is capable of hybridizing to a region that is preferentially methylated in a neuron cell.

In some embodiments, further including a second plurality of oligonucleotides, wherein each oligonucleotide in the second plurality is capable of hybridizing to a region that is preferentially unmethylated in a neuron cell.

In some embodiments, the kit is for determining efficacy of a potential treatment of a neurodegenerative disease or condition. In some embodiments, the neurodegenerative disease or condition is selected from Alzheimer's disease, Huntington disease, Parkinson's disease, amyotrophic lateral sclerosis (ALS), ataxia, multiple sclerosis, multiple system atrophy, or concussion. In some embodiments, the neuron is a motor neuron, a sensory neuron, an interneuron, a dopaminergic neuron, cholinergic neuron, a GABAergic neuron, a glutamatergic neuron, or a cortical neuron. In some embodiments, the neuron is from the forebrain, midbrain, or hindbrain. In some embodiments, the neuron is from the frontal lobe, temporal lobe, parietal lobe, occipital lobe, cerebellum, or brain stem.

In some embodiments, the plurality of oligonucleotides includes one or more oligonucleotides having a nucleotide sequence of any one of SEQ ID NOs: 3-6.

In another aspect, the present disclosure provides a method of detecting cell-free DNA from neurons in a blood sample, including: (i) obtaining cell-free DNA from a blood sample from a human subject; and (ii) detecting whether cell-free DNA from neurons is present in the blood sample by methylation analysis comprising subjecting the cell-free DNA from the blood sample to sequencing of whole amplicons of DNA.

In some embodiments, the whole amplicons are at least about 50 base pairs (bp) in length. In some embodiments, the whole amplicons are between about 50 and about 500 base pairs in length.

In some embodiments, the whole amplicons were produced using one or more primers that include the sequence of any one of SEQ ID NOs: 3-6. In some embodiments, the whole amplicons were produced using one or more primers targeting a region selected from the regions listed in Table 1.

In another aspect, the present disclosure provides a method for determining the methylation status of an amplicon, including: (i) obtaining cell-free DNA from a blood sample from a human subject; (ii) converting 5-methylcytosine in the cell-free DNA to a different nucleotide, thereby producing converted cell-free DNA; (iii) amplifying the converted cell-free DNA, thereby producing an amplicon; and (iv) sequencing the amplicon, wherein the amplicon is between about 50 bp and about 500 bp in length.

In some embodiments, the whole amplicons were produced using one or more primers that include the sequence of any one of SEQ ID NOs: 3-6. In some embodiments, the whole amplicons were produced using one or more primers targeting a region selected from the regions listed in Table 1.

### DETAILED DESCRIPTION

After reading this description it will become apparent to one skilled in the art how to implement the present disclosure in various alternative embodiments and alternative applications. However, all the various embodiments of the present invention will not be described herein. It will be understood that the embodiments presented here are presented by way of an example only, and not limitation. As such, this detailed description of various alternative embodiments should not be construed to limit the scope or breadth of the present disclosure as set forth herein.

Before the present technology is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

The detailed description divided into various sections only for the reader's convenience and disclosure found in any section may be combined with that in another section. Titles or subtitles may be used in the specification for the convenience of a reader, which are not intended to influence the scope of the present disclosure.

### Definitions

Unless defined otherwise, all technical and scientific terms used herein have the same meaning as commonly understood by one of ordinary skill in the art to which this disclosure belongs. In this specification and in the claims that follow, reference will be made to a number of terms that shall be defined to have the following meanings:

The terminology used herein is for the purpose of describing particular embodiments only and is not intended to be limiting. As used herein, the singular forms “a”, “an” and “the” are intended to include the plural forms as well, unless the context clearly indicates otherwise.

“Optional” or “optionally” means that the subsequently described event or circumstance can or cannot occur, and that the description includes instances where the event or circumstance occurs and instances where it does not.

The term “about” when used before a numerical designation, e.g., temperature, time, amount, concentration, and such other, including a range, indicates approximations which may vary by (+) or (−) 10%, 5%, 1%, or any subrange or subvalue there between. Preferably, the term “about” when used with regard to an amount means that the amount may vary by +/−10%.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this disclosure.

The term “cell-free DNA” as used herein refers to short fragments of DNA released into the bloodstream through cell death.

The term “neuron” as used herein refers to any neuron or neuronal cell type. By way of non-limiting example, a neuron may be a motor neuron, a dopaminergic neuron, a cortical neuron, a sensory neuron, an interneuron, or medium spiny neuron.

### Methods

Due to the inherent complications surrounding biopsies secured from brain tissue, other tissues must be used when diagnosing diseases in the brain. Previous work using DNA methylation signatures as a possible neurodegenerative diagnostic has been attempted using cerebral spinal fluid as the primary liquid biopsy. CSF fluid is extracted using a spinal tap. A painful expensive process that requires a medical degree and includes many potential health risks to the patient. The advantage of the disclosed method is that it can be performed on a blood sample, a tissue that has become a standard liquid biopsy in health care as the extraction thereof is relatively simple, inexpensive, and entails low risk for the patient.

This disclosure relates to a blood based two-part diagnostic tool designed to diagnose pre-symptomatic neurodegenerative disease or condition. Part one consists of an assay designed to amplify and sequence pre-specified regions of cell-free DNA. Part two consists of a computer-implemented analysis (e.g., python-derived) that analyzes methylation signatures to identify neuronal DNA and provide a diagnosis of pre-symptomatic neurodegenerative disease or condition.

Using standard protocols for blood-based cell-free DNA extraction, DNA is then bisulfite converted and amplified at pre-selected sites of the genome using PCR. The assay used for PCR amplification is a uniquely designed assay meant to target and amplify the preselected regions of bisulfite converted DNA. These pre-selected regions contain methylation signatures unique to neuron cells, which allows for easy identification of neuronal-derived DNA from the DNA of all other cell types likely to be found in cell free DNA of the blood. Following amplification, regions are then sequenced via DNA sequencing.

The methylation signatures of each single read are then analyzed. This is done using previously established methylation “blueprints” for neuronal cells and whole blood cells. The computer-implemented analysis of the disclosed invention compares each read to these “blueprints” and predicts which cell type each read is derived from. Once neuronal DNA is identified the analysis then predicts pre-symptomatic neurodegenerative disease or condition. This is done by first analyzing the amount of neuronal cell free DNA present in the blood, and second by comparing methylation signatures to methylation “blueprints” of patients with the neurodegenerative disease or condition. The combination of both ultimately provides a diagnosis of whether or not the patient has pre-symptomatic neurodegenerative disease or condition.

Cell types, such as neurons, have distinct methylation patterns (“blueprints”) that can be used to differentiate them from other cell types. In addition, the methylation pattern can help to distinguish individual neuronal types from other neuronal types, for example to differentiate between a motor neuron and a dopaminergic neuron. Cell-free DNA, such as that found in the blood, is a result of cell death throughout the body. The inventors have discovered that analyzing cell-free DNA for methylation patterns from neurons can predict the presence of a neurodegenerative disease or condition in a subject.

This method can be used to detect neurodegenerative disease or condition before the on-set of symptoms or in early stages when symptoms are mild. The method can also be used to determine the incidence, severity and/or duration (e.g., resolution) of a traumatic brain injury, such as concussion. Still further, the method can be used to distinguish between different types of neurodegenerative diseases or conditions. In additional aspects, the method can be used to evaluate efficacy of a therapeutic agent for treatment of a neurodegenerative disease or condition.

In some aspects, a method of treatment of a subject who has or is at risk of having a neurodegenerative disease or condition is provided. In embodiments, the method includes:
(i) obtaining cell-free DNA from a blood sample from the subject;
(ii) analyzing a methylation pattern of the cell-free DNA;
(iii) determining a percentage of cell-free DNA from neurons;
(iv) comparing the percentage of cell-free DNA from neurons to a control; and
(v) administering a therapeutic agent that treats or prevents the neurodegenerative disease or condition when the percentage of cell-free DNA from neurons is greater than the control.

In embodiments, step (ii) includes analyzing a methylation pattern of whole amplicons of DNA. In embodiments, the whole amplicons are at least about 50 base pairs (bp) in length.

In embodiments, the whole amplicons are between about 50 and about 500 base pairs in length. In embodiments, the whole amplicons are between about 60 and about 500 base pairs in length. In embodiments, the whole amplicons are between about 70 and about 500 base pairs in length. In embodiments, the whole amplicons are between about 80 and about 500 base pairs in length. In embodiments, the whole amplicons are between about 90 and about 500 base pairs in length. In embodiments, the whole amplicons are between about 100 and about 500 base pairs in length. In embodiments, the whole amplicons are between about 150 and about 500 base pairs in length. In embodiments, the whole amplicons are between about 200 and about 500 base pairs in length. In embodiments, the whole amplicons are between about 250 and about 500 base pairs in length. In embodiments, the whole amplicons are between about 300 and about 500 base pairs in length. In embodiments, the whole amplicons are between about 350 and about 500 base pairs in length. In embodiments, the whole amplicons are between about 400 and about 500 base pairs in length. In embodiments, the whole amplicons are between about 450 and about 500 base pairs in length.

In embodiments, the whole amplicons are between about 50 and about 450 base pairs in length. In embodiments, the whole amplicons are between about 50 and about 400 base pairs in length. In embodiments, the whole amplicons are between about 50 and about 350 base pairs in length. In embodiments, the whole amplicons are between about 50 and about 300 base pairs in length. In embodiments, the whole amplicons are between about 50 and about 250 base pairs in length. In embodiments, the whole amplicons are between about 50 and about 200 base pairs in length. In embodiments, the whole amplicons are between about 50 and about 150 base pairs in length. In embodiments, the whole amplicons are between about 50 and about 100 base pairs in length. In embodiments, the whole amplicons are between about 50 and about 90 base pairs in length. In embodiments, the whole amplicons are between about 50 and about 80 base pairs in length. In embodiments, the whole amplicons are between about 50 and about 70 base pairs in length. In embodiments, the whole amplicons are between about 50 and about 60 base pairs in length. Whole amplicon length may be any value or subrange within recited ranges, including endpoints.

In some aspects, this disclosure relates to a method of treatment of a subject, wherein the subject has or is at risk of having a neurodegenerative disease or condition, the method including:
(i) obtaining cell-free DNA from a blood sample from the subject;
(ii) analyzing a methylation pattern of the cell-free DNA;
(iii) determining a percentage of cell-free DNA from neurons; and
(iv) administering a therapeutic agent that treats or prevents the neurodegenerative disease or condition when the percentage of cell-free DNA from neurons is greater than about 5%.

In embodiments, the therapeutic agent is administered when the percentage of cell-free DNA from neurons is between about 3% and about 99%. In embodiments, the therapeutic agent is administered when the percentage of cell-free DNA from neurons is between about 3% and about 90%. In embodiments, the therapeutic agent is administered when the percentage of cell-free DNA from neurons is between about 3% and about 80%. In embodiments, the therapeutic agent is administered when the percentage of cell-free DNA from neurons is between about 3% and about 70%. In embodiments, the therapeutic agent is administered when the percentage of cell-free DNA from neurons is between about 3% and about 60%. In embodiments, the therapeutic agent is administered when the percentage of cell-free DNA from neurons is between about 3% and about 50%. In embodiments, the therapeutic agent is administered when the percentage of cell-free DNA from neurons is between about 3% and about 40%. In embodiments, the therapeutic agent is administered when the percentage of cell-free DNA from neurons is between about 3% and about 30%. In embodiments, the therapeutic agent is administered when the percentage of cell-free. DNA from neurons is between about 3% and about 20%.

In embodiments, the therapeutic agent is administered when the percentage of cell-free DNA from neurons is greater than about 2%. In embodiments, the therapeutic agent is administered when the percentage of cell-free DNA from neurons is greater than about 3%. In embodiments, the therapeutic agent is administered when the percentage of cell-free DNA from neurons is greater than about 4%. In embodiments, the therapeutic agent is administered when the percentage of cell-free DNA from neurons is greater than about 5%. In embodiments, the therapeutic agent is administered when the percentage of cell-free DNA from neurons is greater than about 6%. In embodiments, the therapeutic agent is administered when the percentage of cell-free DNA from neurons is greater than about 7%. In embodiments, the therapeutic agent is administered when the percentage of cell-free DNA from neurons is greater than about 8%. In embodiments, the therapeutic agent is administered when the percentage of cell-free DNA from neurons is greater than about 9%. In embodiments, the therapeutic agent is administered when the percentage of cell-free DNA from neurons is greater than about 10%. In embodiments, the therapeutic agent is administered when the percentage of cell-free DNA from neurons is greater than about 11%. In embodiments, the therapeutic agent is administered when the percentage of cell-free DNA from neurons is greater than about 12%. In embodiments, the therapeutic agent is administered when the percentage of cell-free DNA from neurons is greater than about 13%. In embodiments, the therapeutic agent is administered when the percentage of cell-free DNA from neurons is greater than about 14%. In embodiments, the therapeutic agent is administered when the percentage of cell-free DNA from neurons is greater than about 15%. In embodiments, the therapeutic agent is administered when the percentage of cell-free DNA from neurons is greater than about 20%.

In some aspects, this disclosure relates to a method of selecting a patient for treatment with a therapeutic agent for treatment of a neurodegenerative disease or condition, the method comprising:
(i) obtaining cell-free DNA from a blood sample from the subject;
(ii) analyzing a methylation pattern of the cell-free DNA;
(iii) determining a percentage of cell-free DNA from neurons; and
(iv) comparing the percentage of cell-free DNA from neurons to a control; wherein the patient is selected for treatment when the percentage of cell-free DNA from neurons is greater than the control.

In embodiments, the patient is selected for treatment when the percentage of cell-free DNA from neurons is between about 3% and about 99%. In embodiments, the patient is selected for treatment when the percentage of cell-free DNA from neurons is between about 3% and about 90%. In embodiments, the patient is selected for treatment when the percentage of cell-free DNA from neurons is between about 3% and about 80%. In embodiments, the patient is selected for treatment when the percentage of cell-free DNA from neurons is between about 3% and about 70%. In embodiments, the patient is selected for treatment when the percentage of cell-free DNA from neurons is between about 3% and about 60%. In embodiments, the patient is selected for treatment when the percentage of cell-free DNA from neurons is between about 3% and about 50%. In embodiments, the patient is selected for treatment when the percentage of cell-free DNA from neurons is between about 3% and about 40%. In embodiments, the patient is selected for treatment when the percentage of cell-free DNA from neurons is between about 3% and about 30%. In embodiments, the patient is selected for treatment when the percentage of cell-free. DNA from neurons is between about 3% and about 20%.

In embodiments, the patient is selected for treatment when the percentage of cell-free DNA from neurons is greater than about 3%. In embodiments, the patient is selected for treatment when the percentage of cell-free DNA from neurons is greater than about 5%. In embodiments, the patient is selected for treatment when the percentage of cell-free DNA from neurons is greater than about 6%. In embodiments, the patient is selected for treatment when the percentage of cell-free DNA from neurons is greater than about 7%. In embodiments, the patient is selected for treatment when the percentage of cell-free DNA from neurons is greater than about 8%. In embodiments, the patient is selected for treatment when the percentage of cell-free DNA from neurons is greater than about 9%. In embodiments, the therapeutic agent is administered when the percentage of cell-free DNA from neurons is greater than about 10%. In embodiments, the patient is selected for treatment when the percentage of cell-free DNA from neurons is greater than about 11%. In embodiments, the patient is selected for treatment when the percentage of cell-free DNA from neurons is greater than about 12%. In embodiments, the patient is selected for treatment when the percentage of cell-free DNA from neurons is greater than about 13%. In embodiments, the patient is selected for treatment when the percentage of cell-free DNA from neurons is greater than about 14%. In embodiments, the patient is selected for treatment when the percentage of cell-free DNA from neurons is greater than about 15%. In embodiments, the patient is selected for treatment when the percentage of cell-free DNA from neurons is greater than about 20%.

In embodiments, a percentage of cell-free DNA from neurons greater than the control indicates an increased risk of neurodegenerative disease or traumatic brain injury.

In some aspects, this disclosure relates to a method of analyzing a biological sample of a subject. In embodiments, the subject has or is at risk of having a neurodegenerative disease or condition. In embodiments, the method includes:
(i) obtaining cell-free DNA from a blood sample from the subject;
(ii) analyzing a methylation pattern of the cell-free DNA;
(iii) determining a percentage of cell-free DNA from neurons; and
(iv) comparing the percentage of cell-free DNA from neurons to a control.

In some aspects, this disclosure relates to a computer product comprising a non-transitory computer readable medium storing a plurality of instructions that when executed control a computer system to analyze a biological sample from a subject to determine the risk of neurodegenerative disease or condition in the subject, the biological sample comprising cell-free DNA. In embodiments, the instructions include:
(i) identifying a first DNA methylation pattern that occurs in a neuron at a rate above a threshold, wherein the first DNA methylation pattern comprises methylation at one or more methylated regions and optionally comprises no methylation at one or more unmethylated regions;
(ii) analyzing a second DNA methylation pattern of the cell-free DNA; and
(iii) computing a relative abundance of the one or more methylated regions and optionally the one or more unmethylated regions in the cell-free DNA; and
(iv) determining the risk of neurodegenerative disease or condition in the subject by comparing the relative abundance to a control.

In some aspects, this disclosure relates to a computer-implemented method of analyzing a biological sample, including:
(i) identifying a first DNA methylation pattern that occurs in a neuron at a rate above a threshold, wherein the first DNA methylation pattern comprises methylation at one or more methylated regions and optionally comprises no methylation at one or more unmethylated regions;
(ii) analyzing a second DNA methylation pattern of the cell-free DNA;
(iii) computing a relative abundance of the one or more methylated regions and optionally the one or more unmethylated regions in the cell-free DNA; and
(iv) determining the risk of neurodegenerative disease or condition in the subject by comparing the relative abundance to a control.

In embodiments, a percentage of cell-free DNA from neurons between about 3% and about 99% indicates an increased risk of neurodegenerative disease or traumatic brain injury. In embodiments, a percentage of cell-free DNA from neurons between about 3% and about 90% indicates an increased risk of neurodegenerative disease or traumatic brain injury. In embodiments, a percentage of cell-free DNA from neurons between about 3% and about 80% indicates an increased risk of neurodegenerative disease or traumatic brain injury. In embodiments, a percentage of cell-free DNA from neurons between about 3% and about 70% indicates an increased risk of neurodegenerative disease or traumatic brain injury. In embodiments, a percentage of cell-free DNA from neurons between about 3% and about 60% indicates an increased risk of neurodegenerative disease or traumatic brain injury. In embodiments, a percentage of cell-free DNA from neurons between about 3% and about 50% indicates an increased risk of neurodegenerative disease or traumatic brain injury. In embodiments, a percentage of cell-free DNA from neurons between about 3% and about 40% indicates an increased risk of neurodegenerative disease or traumatic brain injury. In embodiments, a percentage of cell-free DNA from neurons between about 3% and about 30% indicates an increased risk of neurodegenerative disease or traumatic brain injury. In embodiments, a percentage of cell-free DNA from neurons between about 3% and about 20% indicates an increased risk of neurodegenerative disease or traumatic brain injury.

In embodiments, a percentage of cell-free DNA from neurons greater than about 3% indicates an increased risk of neurodegenerative disease or traumatic brain injury. In embodiments, a percentage of cell-free DNA from neurons greater than about 4% indicates an increased risk of neurodegenerative disease or traumatic brain injury. In embodiments, a percentage of cell-free DNA from neurons greater than about 5% indicates an increased risk of neurodegenerative disease or traumatic brain injury. In embodiments, a percentage of cell-free DNA from neurons greater than about 6% indicates an increased risk of neurodegenerative disease or traumatic brain injury. In embodiments, a percentage of cell-free DNA from neurons greater than about 7% indicates an increased risk of neurodegenerative disease or traumatic brain injury. In embodiments, a percentage of cell-free DNA from neurons greater than about 8% indicates an increased risk of neurodegenerative disease or traumatic brain injury. In embodiments, a percentage of cell-free DNA from neurons greater than about 9% indicates an increased risk of neurodegenerative disease or traumatic brain injury. In embodiments, a percentage of cell-free DNA from neurons greater than about 10% indicates an increased risk of neurodegenerative disease or traumatic brain injury. In embodiments, a percentage of cell-free DNA from neurons greater than about 11% indicates an increased risk of neurodegenerative disease or traumatic brain injury. In embodiments, a percentage of cell-free DNA from neurons greater than about 12% indicates an increased risk of neurodegenerative disease or traumatic brain injury. In embodiments, a percentage of cell-free DNA from neurons greater than about 13% indicates an increased risk of neurodegenerative disease or traumatic brain injury. In embodiments, a percentage of cell-free DNA from neurons greater than about 14% indicates an increased risk of neurodegenerative disease or traumatic brain injury. In embodiments, a percentage of cell-free DNA from neurons greater than about 15% indicates an increased risk of neurodegenerative disease or traumatic brain injury. In embodiments, a percentage of cell-free DNA from neurons greater than about 20% indicates an increased risk of neurodegenerative disease or traumatic brain injury.

In embodiments, determining a percentage of cell-free DNA from neurons comprises comparing the methylation pattern of the cell-free DNA to a neuronal DNA methylation pattern, wherein the neuronal DNA methylation pattern includes methylation at one or more methylated regions. In embodiments, the neuronal DNA methylation pattern includes no methylation at one or more unmethylated regions.

Following sequencing and data processing, the methylation state of each molecule may be assessed.

In some embodiments, the sequences from the sample are aligned to a reference DNA sequence. In some embodiments, the reference DNA sequence comprises the human genome. In some embodiments, the reference DNA sequence comprises a portion of the human genome. In some embodiments, the reference DNA sequence comprises the regions amplified by the PCR. In some embodiments, the reference DNA sequence comprises a portion of the PCR product. Following alignment, positions identified as having different methylation states between the tissues can be analyzed to measure the relative frequency of base calls at the position to determine the estimated fraction methylated in the pool. In some embodiments, this fraction methylated at each position can then be analyzed in conjunction with other positions to find the overall methylated state of the sequenced DNA molecule.

In some embodiments, the sequences from the sample are searched to identify particular base sequences of length N within the sequenced DNA molecule, and Nis 2, 3, 4, 5, 6, 7, 8, 9, 10, 11, 12, 13, 14, 15, 16, 17, 18, 19, or 20 bases long. In some embodiments, the base sequence is adjacent to the base being evaluated. In some embodiments, the base sequence encompasses the base being evaluated. In some embodiments, two or more versions of the sequence are used to represent the methylated and unmethylated states of the DNA sequence.

In some embodiments, the sequences from the sample are fed into a machine-learning algorithm. In some embodiments, the machine-learning algorithm comprises a neural network. In some embodiments, the machine-learning algorithm comprises a multi-layer perceptron. In some embodiments, the machine-learning algorithm comprises a convolutional neural network. In some embodiments, the machine-learning algorithm uses base-called DNA sequences as input. In some embodiments, the DNA sequence is broken down into k-mers; and k=2, 3, 4, 5, 6, 7, 8, 9, 10, 11, 12, 13, 14, 15, 16, 17, 18, 19, or 20 bases long. For example, in one embodiment, the sequencing data is broken in overlapping hexamers (k=6). These hexamers were counted and fed into a multi-layer perceptron comprising 4096 input neurons (one for every possible hexamer sequence), 8 neurons in the hidden layer and 1 output neuron that classified the reads as methylated or non-methylated. The model was trained using 100,000 reads selected from purified tissue samples containing exclusively methylated or non-methylated DNA at the locus examined. Cross-validation using 20% of the data showed a final accuracy of 99.8% and a loss of 0.01.

The control may be any suitable control. In embodiments, the control is a percentage of cell-free DNA from neurons in a blood sample from an untreated subject. In embodiments, the control is a percentage of cell-free DNA from neurons in a blood sample from a healthy subject. In embodiments, the control is a blood sample from the subject(s) prior to treatment. In embodiments, the control is a threshold. In embodiments, the threshold is a percentage of cell-free DNA from neurons greater than about 3%. In embodiments, the threshold is a percentage of cell-free DNA from neurons greater than about 4%. In embodiments, the threshold is a percentage of cell-free DNA from neurons greater than about 5%. In embodiments, the threshold is a percentage of cell-free DNA from neurons greater than about 6%. In embodiments, the threshold is a percentage of cell-free DNA from neurons greater than about 7%. In embodiments, the threshold is a percentage of cell-free DNA from neurons greater than about 8%. In embodiments, the threshold is a percentage of cell-free DNA from neurons greater than about 9%. In embodiments, the threshold is a percentage of cell-free DNA from neurons greater than about 10%. In embodiments, the threshold is a percentage of cell-free DNA from neurons greater than about 11%. In embodiments, the threshold is a percentage of cell-free DNA from neurons greater than about 12%. In embodiments, the threshold is a percentage of cell-free DNA from neurons greater than about 13%. In embodiments, the threshold is a percentage of cell-free DNA from neurons greater than about 14%. In embodiments, the threshold is a percentage of cell-free DNA from neurons greater than about 15%. In embodiments, the threshold is a percentage of cell-free DNA from neurons greater than about 20%.

In embodiments, the neurodegenerative disease is Alzheimer's disease. In embodiments, the neurodegenerative disease is Huntington disease. In embodiments, the neurodegenerative disease is Parkinson's disease. In embodiments, the neurodegenerative disease is amyotrophic lateral sclerosis (ALS). In embodiments, the neurodegenerative disease is ataxia. In embodiments, the neurodegenerative disease is multiple sclerosis. In embodiments, the neurodegenerative disease or condition is multiple system atrophy. In embodiments, the neurodegenerative condition is mild traumatic brain injury. In embodiments, the neurodegenerative condition is concussion. One or more of these diseases or conditions may be expressly excluded.

The neuron(s) may be any neuron type, or combination thereof. In embodiments, the neuron is a motor neuron. In embodiments, the neuron is a sensory neuron. In embodiments, the neuron is an interneuron. In embodiments, the neuron is a dopaminergic neuron. In embodiments, the neuron is a GABAergic neuron. In embodiments, the neuron is a glutamatergic neuron. In embodiments, the neuron is a cortical neuron.

In embodiments, the neuron is from the forebrain. In embodiments, the neuron is from the midbrain. In embodiments, the neuron is from the hindbrain. In embodiments, the neuron is from the frontal lobe. In embodiments, the neuron is from the temporal lobe. In embodiments, the neuron is from the parietal lobe. In embodiments, the neuron is from the occipital lobe. In embodiments, the neuron is from the cerebellum. In embodiments, the neuron is from the brain stem.

In embodiments, analyzing a DNA methylation pattern includes determining the methylation status of a region of DNA. In embodiments, the DNA is chromosomal DNA. In embodiments, the region is a region that is preferentially methylated (more likely to be methylated) in a neuron or a type of neuron compared to one or more other cell types. In embodiments, the region is a region that is preferentially unmethylated (more likely to be unmethylated) in a neuron or a type of neuron compared to one or more other cell types.

In embodiments, the methylation status of one or more regions set forth in Table 1 is analyzed. In embodiments, the methylation status of one or more subregions within one or more regions set forth in Table 1 is analyzed. In embodiments, the methylation status of chr3:42190679,42191148 is analyzed. In embodiments, an increased percentage (compared to a control) of cfDNA having methylation in one or more regions set forth in Table 1 (or one or more subregions within one or more regions set forth in Table 1) indicates an increased risk of Alzheimer's disease. In embodiments, an increased percentage (compared to a control) of cfDNA having no methylation in one or more regions set forth in Table 1 (or one or more subregions within one or more regions set forth in Table 1) indicates an increased risk of Alzheimer's disease.

TABLE 1
Chromosome Regions - Alzheimer's Disease

| | | |
|---|---|---|
| chr12_118627943_118627944 | chr7_27152583_27156063 | chr1_37773813_37773904 |
| chr5_141225619_141225620 | chr5_107205065_107205066 | chr6_97754012_97754013 |
| chr5_124077966_124077967 | chr11_47554407_47554408 | chr11_122179528_122179529 |
| chr19_3507867_3507868 | chr1_243877024_243877448 | chr2_120014005_120014006 |
| chr17_46131583_46131584 | chr4_48656001_48656002 | chr11_13303971_13303972 |
| chr16_12189952_12189953 | chr4_90675036_90675037 | chr1_244219887_244219888 |
| chr1_67001108_67001109 | chr1_8658581_8658582 | chr17_64451448_64451449 |
| chr8_26440349_26440350 | chr17_36511291_36511292 | chr4_25818940_25818941 |
| chr1_160180434_160180435 | chr4_8508604_8508605 | chr20_47862976_47862977 |
| chr7_127584444_127584445 | chr10_51589507_51589508 | chr1_77904032_77904033 |
| chr21_42215594_42215595 | chr5_149765871_149765872 | chr5_27038605_27038837 |
| chr11_65066368_65066369 | chr12_96628738_96628739 | chr2_109752622_109752623 |
| chr2_26200001_26200002 | chr16_87429184_87429185 | chr15_40268421_40269215 |
| chr8_131011240_131011241 | chrX_103085819_103085820 | chr10_33671135_33671136 |
| chr3_42190778_42191048 | chr14_74184843_74186280 | chr19_54055365_54055366 |
| chr2_201245077_201245078 | chr5_6452065_6452066 | chr11_70557445_70557882 |
| chr5_147160551_147160552 | chr11_105483680_105483681 | chr7_151593084_151593085 |
| chr1_206135024_206135025 | chr10_11056954_11056955 | chr2_161920006_161920007 |
| chr7_44360795_44360796 | chr10_80981119_80981130 | chr8_881586_881587 |
| chr16_87465786_87465787 | chr12_14109211_14109585 | chr2_2328391_2328710 |
| chr2_110064019_110064020 | chr17_26267036_26267037 | chr7_128395929_128395930 |
| chr1_159111423_159111424 | chr5_14744206_14744207 | chr1_184003184_184003185 |
| chr3_9809922_9809923 | chr17_29740988_29740989 | chr9_127047671_127047672 |
| chr11_84844436_84844437 | chr14_50576289_50576290 | chr8_10253076_10253077 |
| chr10_4386802_4386803 | chr11_123396039_123396594 | chr16_30136024_30136025 |
| chr7_107110551_107110552 | chr8_119626989_119626990 | chr11_122156457_122156458 |
| chr10_112580109_112580110 | chr10_15265252_15265253 | chr2_236300720_236300938 |
| chr2_47236103_47236104 | chr1_203567067_203567068 | chr4_7632386_7632387 |
| chr9_82278827_82278828 | chr1_109260678_109260679 | chr1_112960391_112960392 |
| chr5_139626136_139626137 | chr6_107506867_107506868 | chr11_117232202_117232455 |
| chr12_110154229_110154230 | chr4_144275126_144275127 | chr1_164543254_164543255 |
| chr1_214834360_214834361 | chr5_11906843_11906844 | chr8_22860884_22860885 |
| chr19_47107173_47107174 | chr8_18743898_18744447 | chr8_10268916_10268917 |
| chr17_63634484_63634485 | chr4_141207794_141207853 | chr12_120130253_120130647 |
| chr19_2654246_2654247 | chr2_217232992_217232993 | chr17_35017872_35017917 |
| chr17_29624227_29624440 | chr4_160204019_160204020 | chr6_138752062_138752063 |
| chr14_77403711_77403712 | chr6_166117509_166117677 | chr19_31765880_31765881 |
| chr8_9537322_9537323 | chr3_35683815_35683820 | chr9_132948449_132948450 |
| chr5_138441071_138441072 | chr10_70362422_70362423 | chr4_159442782_159442783 |
| chr21_17961737_17962125 | chr6_13248958_13248959 | chr16_12272324_12272325 |
| chr17_38447008_38447009 | chr11_63647865_63647866 | chr7_140776653_140776654 |
| chr3_16558965_16558966 | chr1_9904386_9904387 | chr1_8430745_8430746 |
| chr12_118471960_118471961 | chr5_138061348_138061349 | chr3_188607928_188607929 |
| chr17_43318045_43319383 | chr19_36490314_36490315 | chr19_575412_576048 |
| chr1_156564330_156564331 | chrX_53115787_53115788 | chr6_34356253_34356254 |
| chr1_42886004_42886005 | chr4_16731545_16731546 | chr15_73975176_73975177 |
| chr3_52056831_52056832 | chr2_204427199_204427200 | chr18_43917496_43917497 |
| chr4_89679571_89679572 | chr10_62241191_62241192 | chr1_244216231_244218538 |
| chr6_5152468_5152469 | chr2_234913188_234913189 | chr8_124656433_124656434 |
| chr2_136343909_136343910 | chr14_91017836_91017837 | chr14_77577612_77577613 |
| chr14_23982474_23982530 | chr6_85824168_85824217 | chr10_128250156_128250157 |
| chr21_17441953_17442804 | chr1_4468051_4468090 | chr17_61046563_61046564 |
| chr8_101522535_101522536 | chr1_60282400_60282401 | chr19_15563870_15564106 |
| chr1_82147424_82147425 | chr2_217205778_217205779 | chr2_166535708_166535709 |
| chr3_13420826_13420962 | chr16_11697522_11697523 | chr6_139293014_139293015 |
| chr12_122891696_122891697 | chr18_42323716_42325087 | chr11_73053649_73054402 |
| chr2_131814000_131814001 | chr5_179751794_179751795 | chr15_63034966_63034967 |
| chr14_74968209_74968210 | chr11_63466987_63466988 | chr11_36599110_36599111 |
| chr5_138205434_138205435 | chr6_53851924_53851925 | chr4_802229_803015 |
| chr12_99146745_99146746 | chr6_113932902_113932903 | chr8_134044590_134044591 |
| chr1_61649907_61649974 | chr3_10140678_10140679 | chr6_52382441_52382442 |
| chr20_10202522_10202523 | chr16_65104932_65105715 | chr13_73438525_73438526 |
| chr11_72347339_72347340 | chr1_240078452_240078453 | chr5_17238232_17238233 |
| chr2_60804996_608049970 | chr19_40872963_40872964 | chr3_71158765_71158766 |
| chr7_150896413_150896490 | chr17_79856430_79856431 | chr17_53363128_53363129 |
| chr3_114958699_114958700 | chr2_45986586_45986587 | chr15_47676823_47676824 |
| chr8_104831516_104831517 | chr12_51901293_51901294 | chr5_102710400_102710401 |
| chr10_103090818_103090819 | chr18_24050283_24050284 | chr2_173995795_173995796 |
| chr4_48915431_48915432 | chr7_32099501_32099502 | chr12_123491781_123491782 |
| chr1_201665422_201665423 | chr5_155901557_155901558 | chr7_111396894_111396895 |
| chr12_120241287_120242514 | chr10_10337051_10337313 | chr12_130976260_130976552 |
| chr9_96846674_96846725 | chr2_9511523_9511524 | chr6_160510345_160510346 |
| chr14_69015199_69015200 | chr11_46005268_46005269 | chr5_156955280_156955281 |
| chr12_94580329_94580932 | chr10_38144887_38144888 | chr1_53600741_53600890 |
| chr1_151035398_151035399 | chr13_31609975_31609976 | chr1_42138658_42138659 |
| chr1_89993815_89993816 | chr6_37276086_37276087 | chr3_124904931_124904932 |
| chr17_30842946_30842947 | chr2_231555187_231555188 | chr5_123121409_123121410 |
| chr19_40873973_40873974 | chr12_4596426_4596427 | chr6_90385873_90385874 |
| chr1_21993401_21993556 | chr1_243584668_243584862 | chr2_71791254_71791255 |
| chr11_1886783_1886800 | chr9_132900904_132900905 | chr19_47113209_47113223 |
| chr11_62790783_62790784 | chr2_135218333_135218334 | chr14_55740496_55740497 |
| chr3_155394109_155394429 | chr11_17849903_17849904 | chr20_62722933_62722934 |
| chr16_87384599_87384764 | chr9_138008934_138008935 | chr2_208091961_208091962 |
| chr1_160189219_160189220 | chr13_109984684_109984685 | chr3_132760544_132760545 |
| chr10_405182_405363 | chr4_148861409_148861410 | chr1_27893886_27893926 |
| chr9_35539509_35539510 | chr7_51376950_51376951 | chr5_16913076_16913077 |
| chr10_14447185_14447186 | chr3_129383357_129383358 | chr4_116034871_116035233 |
| chr16_72232102_72232103 | chr16_69098945_69098946 | chr4_87989696_87989697 |
| chr7_158207371_158207372 | chr16_78218445_78218446 | chr11_43852883_43852884 |
| chr13_50975679_50975680 | chr16_23420385_23420568 | chr5_140903294_140903295 |
| chr1_11979188_11979189 | chr6_84761960_84761961 | chr18_12646025_12646026 |
| chr8_135634128_135634129 | chr2_23879137_23879138 | chr17_45699260_45699323 |
| chr1_201794425_201794426 | chr7_130673538_130673539 | chr1_229618011_229618012 |
| chr2_161980152_161980153 | chr15_63220652_63220653 | chr3_186659238_186659239 |
| chr10_34405759_34405760 | chr18_42321831_42321832 | chr12_114337676_114337928 |
| chr6_22148738_22148739 | chr2_54292798_54292799 | chr3_13597750_13597751 |
| chr6_16804166_16804167 | chr11_45109122_45109123 | chr17_78765948_78765949 |
| chr10_35698554_35698555 | chr1_183578811_183578812 | chr20_6514844_6514845 |
| chr5_173390427_173390428 | chr4_176735244_176735245 | chr4_57998425_57998426 |
| chr19_19639553_19640076 | chr4_186876457_186876458 | chr21_42217001_42217002 |
| chr19_19104064_19104065 | chr3_44140250_44140251 | chr4_62606570_62606571 |
| chr13_32835962_32835963 | chr11_6435089_6435090 | chr2_9816010_9816011 |
| chr16_79030857_79030891 | chr2_70960959_70960960 | chr1_169088807_169088808 |
| chr4_72246676_72246677 | chr5_115697214_115697697 | chr3_42094749_42094750 |
| chr12_26386833_26386834 | chr1_4794072_4795115 | chr17_42196657_42196658 |
| chr12_125500511_125500701 | chr1_211765568_211765569 | chr2_203294144_203294145 |
| chr19_15568360_15568936 | chr17_65527566_65527567 | chr8_131374842_131374843 |
| chr4_87278335_87278336 | chr14_103979422_103979423 | chr5_74913041_74913042 |
| chr16_27063429_27063430 | chr10_1686427_1686471 | chr12_92422572_92422573 |
| chr1_6336519_6336712 | chrX_62972602_62972603 | chr16_73265382_73266786 |
| chr4_113269890_113269891 | chr11_84028382_84028583 | chr11_45306397_45306398 |
| chr2_109907962_109907963 | chr6_136672985_136672986 | chr4_122706839_122706840 |
| chr8_10210267_10210283 | chr16_12637499_12637500 | chr4_17060364_17060365 |
| chr1_169072106_169072107 | chr6_16965070_16965071 | chr5_175257943_175257944 |
| chr7_127663097_127663098 | chr10_93100919_93100920 | chr10_5986488_5986489 |
| chr1_244545617_244545618 | chr1_43145343_43145344 | chr2_11059462_11059463 |
| chr21_27540106_27540729 | chr16_28193063_28193064 | chr7_157140029_157140030 |
| chr17_49122713_49122714 | chr17_42164433_42164434 | chr6_157493773_157493774 |
| chr13_45767909_45768902 | chr2_173080153_173080154 | chr17_46627384_46630631 |
| chr4_171030995_171030996 | chr4_186652454_186652455 | chr1_67805528_67805529 |
| chr2_43133914_43133915 | chr2_47420867_47420868 | chr16_66223243_66223244 |
| chr1_111145889_111145890 | chr14_30054523_30054524 | chr12_76114755_76114756 |
| chr1_8433089_8433090 | chr17_9832474_9832475 | chr8_124498970_124498971 |
| chr2_16832247_16832248 | chr16_88677643_88677644 | chr4_21004400_21004401 |
| chr7_705435_705694 | chr7_43912425_43912426 | chr2_202330990_202330991 |
| chr17_49123976_49123977 | chr5_156988400_156988401 | chr1_178341865_178341866 |
| chr6_147361831_147361832 | chr6_88506326_88506327 | chr10_79238225_79238226 |
| chr7_27178861_27179433 | chr5_121499498_121499499 | chr2_129320987_129320988 |
| chr9_134484581_134484582 | chr8_123801352_123801353 | chr19_17942025_17943314 |
| chr1_51887613_51888962 | chr1_26253412_26253413 | chr21_22372231_22373040 |
| chr3_13112502_13112503 | chr1_50801596_50801597 | chr3_120179862_120179863 |
| chr6_30693163_30693164 | chr9_132937709_132937710 | chr11_36131322_36131323 |
| chr12_106507713_106507714 | chr20_32260699_32260700 | chr3_66033260_66033261 |
| chr5_57753951_57753952 | chr15_26877820_26877821 | chr1_240260050_240260051 |
| chr8_9775751_9775752 | chr8_58982720_58982908 | chr12_57783795_57783796 |
| chr5_71310320_71310321 | chr4_188829287_188829288 | chr10_95518976_95518977 |
| chr1_37760158_37760159 | chr15_40583227_40583423 | chr3_58410836_58410837 |
| chr3_9225565_9225566 | chr17_74416551_74416552 | chr4_147187180_147187181 |
| chr5_143588059_143588060 | chr10_105449972_105449973 | chr11_78402382_78402383 |
| chr11_119542542_119542543 | chr14_89866110_89866111 | chr1_178067721_178067722 |
| chr8_40235245_40235246 | chr2_209054670_209055252 | chr6_11711971_11711972 |
| chr3_24187579_24187580 | chr5_149650243_149650244 | chr2_121467863_121468082 |
| chr19_50550284_50550360 | chr6_41554529_41554544 | chr1_231002232_231002233 |
| chr6_34569886_34569887 | chr14_100485494_100485495 | chr3_4762242_4762243 |
| chr11_8133258_8133259 | chr2_197399559_197399560 | chr6_128388820_128388821 |
| chr14_94179847_94179848 | chr4_114213574_114214450 | chr3_9289747_9289894 |
| chr12_125333267_125333359 | chr13_107142673_107142674 | chr9_132016282_132016283 |
| chr3_149685395_149685396 | chrX_118374789_118374790 | chr4_110583727_110583728 |
| chr16_12209538_12209539 | chr11_82430349_82430350 | chr1_77505574_77505575 |
| chr17_76719591_76719944 | chr6_90271866_90271867 | chr1_42115829_42115830 |
| chr10_75873408_75873409 | chr5_131008111_131008112 | chr12_66034196_66034197 |
| chr12_125621344_125621410 | chr12_67072769_67073761 | chr10_92783852_92783853 |
| chr3_106917496_106917497 | chr10_4540959_4540960 | chr6_25232780_25232875 |
| chr4_170101082_170101083 | chr3_56809497_56810750 | chr6_36761843_36761844 |
| chr12_114328074_114328075 | chr11_82781482_82781483 | chr3_35720528_35721285 |
| chr7_116659609_116660241 | chr16_3824553_3824554 | chr5_73502614_73502615 |
| chr12_1700011_1700012 | chr4_103542236_103542237 | chr3_186211511_186211671 |
| chr6_44194767_44194803 | chr8_121666457_121666458 | chr8_38175544_38175685 |
| chr11_17994507_17994508 | chr2_135985164_135985165 | chr11_45894617_45894618 |
| chr5_7399037_7399038 | chr1_19636065_19636066 | chr2_210290621_210290622 |
| chr1_36317380_36317381 | chr8_143262091_143262573 | chr12_58128450_58133489 |
| chr16_24057138_24057139 | chr5_173054985_173054986 | chr1_116958941_116958942 |
| chr6_5764816_5764817 | chr7_105283982_105283983 | chr11_72501130_72501131 |
| chr9_131317330_131317331 | chr11_8936435_8936436 | chr5_141205206_141205207 |
| chr12_123582277_123582278 | chr5_137790999_137791000 | chr1_116902548_116902549 |
| chr4_114038454_114038455 | chr4_146740968_146740969 | chr2_11546576_11546577 |
| chr14_77491023_77491024 | chr11_74154543_74154544 | chr5_137825623_137825624 |
| chr18_31799784_31799785 | chr5_14440492_14441262 | chr3_140813769_140814134 |
| chr6_148142629_148142630 | chr7_28145849_28145850 | chr16_70804779_70804780 |
| chr15_95713013_95713101 | chr7_75984284_75984285 | chr17_46607103_46608875 |
| chr14_71905595_71905596 | chr3_65387308_65387309 | chr4_6052527_6052555 |
| chr1_26226649_26226650 | chr1_14020212_14020213 | chr11_11417772_11417773 |
| chr11_17928213_17928214 | chr8_131360571_131360572 | chr14_91121401_91121402 |
| chr3_7693146_7693147 | chr15_25787997_25787998 | chr6_11687689_11687690 |
| chr8_131303466_131303467 | chr2_110092156_110092157 | chr6_168064901_168064902 |
| chr10_11046334_11047587 | chr7_102787173_102787747 | chr5_126193470_126193471 |
| chr4_113300446_113300822 | chr1_204799888_204799889 | chr16_70717902_70717903 |
| chr11_123487358_123487359 | chr3_127506604_127506605 | chr5_95744021_95744022 |
| chr12_95671146_95671147 | chr2_4600947_4601054 | chr13_25740027_25740028 |
| chr3_11494984_11494985 | chr5_149673232_149673233 | chr17_35043264_35043265 |
| chr19_50305342_50306073 | chr7_229225_229696 | chr1_181644542_181644543 |
| chr2_207906468_207906469 | chr4_72096973_72096974 | chr14_93175350_93175351 |
| chr9_140304131_140304132 | chr11_118047121_118047204 | chr13_99668212_99668434 |
| chr11_120256570_120256571 | chr12_113215623_113215624 | chr14_93502930_93502931 |
| chr6_5472095_5472096 | chr8_143463227_143463668 | chr1_156387415_156387416 |
| chr15_58782685_58782689 | chr5_150005953_150005954 | chr10_11064196_11064197 |
| chr1_177929192_177929193 | chr5_177753258_177753287 | chr16_644081_644495 |
| chr12_27236803_27236885 | chr15_69841201_69841260 | chr2_149859227_149859733 |
| chr3_14494726_14494727 | chr5_121462562_121462563 | chr11_134479357_134479414 |
| chr16_72459516_72460115 | chr19_19106904_19106905 | chr4_153274105_153274119 |
| chr7_27157818_27157856 | chr5_34553138_34553139 | chr3_124848635_124848636 |
| chr11_129183160_129183283 | chr8_3965218_3965290 | chr5_176898673_176898674 |
| chr2_2335088_2337094 | chr2_50187352_50187353 | chr1_109926115_109926116 |
| chr19_51053313_51053654 | chr11_70328867_70328868 | chr5_172899456_172899457 |
| chr10_95274920_95274921 | chr3_124839640_124839641 | chr4_54548670_54548671 |
| chr2_77747049_77747050 | chr3_141104523_141104524 | chr2_40646370_40646371 |
| chr1_9880771_9880772 | chr2_172175273_172175274 | chr11_61213555_61213556 |
| chr8_29661687_29661688 | chr14_51263199_51263200 | chr2_20504345_20504346 |
| chr15_25930203_25930204 | chrX_109683163_109683491 | chr9_130515119_130517849 |
| chrX_13835198_13835569 | chr11_85422118_85422119 | chr20_62732060_62732061 |
| chr4_154419554_154419555 | chr17_34055277_34055278 | chr2_100498600_100498601 |
| chr5_72950691_72950692 | chr15_29261921_29261922 | chr12_87232511_87232822 |
| chr11_130283454_130283455 | chr8_10030945_10030946 | chr3_127429559_127429560 |
| chr14_67752711_67752712 | chr4_140480051_140480052 | chr3_153778467_153778468 |
| chr4_81120670_81120671 | chr7_110730805_110731528 | chr4_24105842_24105843 |
| chr8_134049349_134049350 | chr4_760039_760040 | chr3_9273803_9273804 |
| chr19_6475456_6477199 | chr4_186877497_186877917 | chr15_51464013_51464014 |
| chr16_67897346_67897347 | chr19_2524064_2524065 | chr4_184908254_184909019 |
| chr19_41246769_41246770 | chr5_174680743_174680744 | chr1_44418720_44418721 |
| chr5_106738731_106738732 | chr1_167604788_167604789 | chr13_107182539_107183636 |
| chr5_43312201_43312202 | chr8_131663355_131663356 | chr8_120499470_120499471 |
| chr3_11655466_11655467 | chr16_81731249_81732039 | chr7_24740509_24740510 |
| chr5_17215163_17215164 | chr11_15220058_15220059 | chr2_165874828_165874829 |
| chr5_123144349_123144350 | chr3_144309719_144309720 | chr14_93899367_93899368 |
| chr3_129407365_129408522 | chr2_102316442_102316497 | chr8_139613712_139614021 |
| chr13_80910763_80911809 | chr10_111728983_111728984 | chr2_12907910_12907914 |
| chr17_30817129_30817398 | chr1_99467969_99467970 | chr2_12674764_12674977 |
| chr14_77587383_77587384 | chr3_49572730_49572731 | chr12_71249040_71249041 |
| chr8_8522725_8522726 | chr12_57946083_57946084 | chr5_74952444_74952445 |
| chr10_105375201_105375202 | chr14_99655593_99655748 | chr20_34025927_34026479 |
| chr5_73969223_73969224 | chr5_124054948_124054949 | chr17_38191472_38191473 |
| chr5_173395358_173395359 | chr1_110122089_110122090 | chr8_94931371_94931372 |
| chr2_169372300_169372301 | chr15_67841407_67841408 | chr11_64405346_64406052 |
| chr3_143316736_143317040 | chr1_234858982_234858983 | chr11_13461802_13461803 |
| chr3_34055800_34055801 | chr1_169078316_169078317 | chr3_13560246_13560247 |
| chr16_23916466_23916467 | chr1_184473341_184473342 | chr14_99681710_99682270 |
| chr8_27591911_27591912 | chr3_185742220_185742221 | chr1_244264748_244264749 |
| chr4_77743172_77743173 | chr11_82745712_82745713 | chr8_67404651_67405743 |
| chr14_24017882_24018056 | chr2_220510231_220510232 | chr4_10076546_10076547 |
| chr17_37350252_37351757 | chr19_47770746_47770747 | chr2_25223659_25223660 |
| chr8_101428643_101428644 | chr3_71251601_71251602 | chr1_246810637_246810638 |
| chrX_21394909_21394910 | chr5_134582868_134582881 | chr3_126554736_126554737 |
| chr1_109772439_109772440 | chr11_65374663_65375705 | chr11_74384522_74384523 |
| chr2_20650578_20650579 | chr9_130027020_130027117 | chr12_130494600_130494698 |
| chr8_18540642_18541989 | chr1_164604806_164604807 | chr5_134608044_134608045 |
| chr5_67585399_67586929 | chr6_152702330_152702661 | chr1_39960691_39960692 |
| chr10_97319133_97319134 | chr12_42000079_42000080 | chr12_116008178_116008259 |
| chr2_54086854_54087553 | chr12_58263161_58263162 | chr17_39067081_39067082 |
| chr4_1845965_1845966 | chr19_1070986_1071623 | chr1_14382172_14382173 |
| chr1_6390053_6390831 | chr16_27526259_27526260 | chr6_21954586_21954587 |
| chr2_173893111_173893112 | chr3_43033200_43033201 | chr14_102438734_102438735 |
| chr18_3845348_3845704 | chr16_87234895_87234896 | chr1_53754097_53754098 |
| chr19_51050258_51050259 | chr11_75132468_75132469 | chr2_110907413_110907414 |
| chr8_40237170_40237171 | chr2_47761452_47761453 | chr17_12199913_12199914 |
| chr17_46700449_46700450 | chr14_40142517_40142518 | chr10_84027418_84027419 |
| chr19_13138816_13138817 | chr2_16245936_16245937 | chr9_84646696_84646697 |
| chr2_46802312_46802313 | chr8_49676562_49676563 | chr9_115926922_115926923 |
| chr2_100188906_100188907 | chr1_241912510_241912769 | chr15_67175702_67175703 |
| chr8_9260335_9260355 | chr3_115618688_115618689 | chr17_72237019_72237020 |
| chr2_145202065_145202066 | chr7_3988772_3989168 | chr1_6113068_6113069 |
| chr7_95818282_95818283 | chr10_63388439_63388440 | chr1_74970626_74970627 |
| chr5_68114337_68114338 | chr6_3154078_3154106 | chr14_91034963_91034964 |
| chr5_173318755_173318756 | chr2_174082101_174082102 | chr11_84634126_84634736 |
| chr3_64149428_64149429 | chr16_27228679_27228680 | chr3_9246501_9246502 |
| chr7_69882555_69882556 | chr17_63186926_63186927 | chr11_132502809_132502810 |
| chr6_53919041_53919042 | chr14_103912507_103912508 | chr10_49552461_49552462 |
| chr17_70339099_70339344 | chr12_24549226_24549227 | chr1_4758079_4758080 |
| chr1_183952796_183952797 | chr5_16794797_16794883 | chr2_55199843_55200202 |
| chr8_131453955_131453956 | chr9_72285588_72285589 | chr2_145260010_145260011 |
| chr1_27384423_27384424 | chr16_86265226_86265227 | chr18_40695592_40696165 |
| chr12_56269767_56269768 | chr11_133970586_133970587 | chr1_7728694_7728695 |
| chr7_44438216_44438217 | chr2_37948491_37948492 | chr10_12680739_12680740 |
| chr7_37123550_37123551 | chr8_43046482_43046483 | chr19_4782235_4782236 |
| chr1_56025098_56025099 | chr11_100463590_100463591 | chr17_46709858_46710002 |
| chr17_1057343_1057542 | chr12_115117351_115117552 | chr2_166491680_166491681 |
| chr1_85151778_85151779 | chr1_164723567_164723568 | chr1_5816986_5817432 |
| chr11_12098045_12098046 | chr3_64193180_64193181 | chr7_105573866_105573867 |
| chr6_3152428_3152429 | chr7_104681396_104681441 | chr4_16679554_16679555 |
| chr15_100077146_100077147 | chrX_47057322_47057323 | chr15_77910606_77910607 |
| chr16_48428016_48428017 | chr1_9868377_9868378 | chr1_11028561_11028562 |
| chr2_183183274_183183275 | chr16_11016097_11016098 | chr1_37513430_37513491 |
| chr4_187572844_187573047 | chr8_133906156_133906157 | chr9_98902324_98902325 |
| chr11_78884803_78884804 | chr4_153527031_153527032 | chr19_5036609_5037220 |
| chr14_51293793_51293794 | chr2_153302353_153302354 | chr13_74315385_74315386 |
| chr14_62343481_62343482 | chr11_17591822_17591874 | chr3_101926162_101926163 |
| chr12_13899195_13899196 | chr20_32269577_32269578 | chr19_36519412_36519413 |
| chr4_134077575_134077576 | chr2_8343710_8343711 | chr17_981518_982155 |
| chr8_25543184_25543203 | chr15_59737729_59737730 | chr4_184882211_184882212 |
| chr16_68440620_68440621 | chr15_45449437_45449438 | chr5_95276578_95276579 |
| chr2_227850069_227850070 | chr12_102821557_102821558 | chr6_116598395_116598396 |
| chr14_76334455_76334456 | chr11_6425978_6425979 | chr7_110722380_110722381 |
| chr18_13264645_13264646 | chr6_12716655_12718531 | chr8_26041003_26041004 |
| chr5_138743684_138743685 | chr15_70699636_70699637 | chr7_51374506_51374507 |
| chr8_79429818_79429819 | chr10_105063519_105063520 | chr7_22232412_22234164 |
| chr11_57532299_57532300 | chr13_45025622_45025623 | chr12_29486854_29486855 |
| chr15_49277592_49277593 | chr12_24381595_24381596 | chr16_87574693_87575345 |
| chr17_77361608_77361668 | chr10_79150517_79150518 | chr17_80648875_80648876 |
| chr19_8590567_8591777 | chr19_15358258_15358259 | chr14_72193535_72193536 |
| chr16_19185373_19185557 | chr2_209029594_209029783 | chr5_6159585_6159586 |
| chr2_193013929_193013930 | chr2_153252785_153252786 | chr3_197081654_197081655 |
| chr7_49560538_49560539 | chr3_61833671_61833672 | chr4_143286770_143286771 |
| chr17_37042201_37042202 | chr5_175236870_175236871 | chr19_871087_871088 |
| chr14_91820590_91820591 | chr6_116550594_116550595 | chr8_22861942_22861943 |
| chr2_47465189_47465190 | chr14_69754550_69754551 | chr14_101538070_101538071 |
| chr10_112582591_112582592 | chr1_36180082_36180083 | chr1_19496406_19496407 |
| chr1_10169029_10169030 | chr18_74798054_74798055 | chr11_8692821_8692822 |
| chr6_166470330_166470331 | chr8_8182003_8182748 | chr8_128956842_128956843 |
| chr5_173342997_173342998 | chr7_130606228_130606500 | chr11_65256983_65256984 |
| chr11_126936873_126936874 | chr11_45373299_45373300 | chr1_6140589_6140590 |
| chr4_154179401_154179402 | chr12_16756662_16756663 | chr3_124531371_124531372 |
| chr17_76713379_76713380 | chr9_137693053_137693278 | chr3_164908619_164908620 |
| chr3_49000524_49000525 | chr19_15360953_15360954 | chr12_54733883_54733884 |
| chr6_155540192_155540193 | chr20_43435091_43435092 | chr11_8679885_8681336 |
| chr15_89924975_89924976 | chr17_79035225_79035419 | chr2_10302391_10302463 |
| chr11_12136405_12136406 | chr8_104117866_104117867 | chr10_64957287_64957288 |
| chr15_74687517_74687518 | chr17_64640562_64640563 | chr13_113698408_113702479 |
| chr2_109812089_109812090 | chr13_35192071_35192072 | chr7_141385142_141385274 |
| chr1_178621037_178621038 | chr11_1421113_1421210 | chr2_76905622_76905623 |
| chr16_84518355_84518356 | chr1_87803705_87803706 | chr3_34884326_34884327 |
| chr5_150861640_150861641 | chr1_21966310_21966501 | chr2_173330296_173330396 |
| chr12_132266778_132266779 | chr8_49804177_49804178 | chr9_138132949_138132950 |
| chr6_24140822_24140900 | chr7_17081421_17081422 | chr6_164572718_164572719 |
| chr5_78807149_78807150 | chr7_101754758_101755187 | chr21_46146433_46146434 |
| chr14_71955729_71955730 | chr15_31597352_31597353 | chr15_92667819_92667820 |
| chr3_123162899_123162900 | chr4_146804010_146804011 | chr16_56821271_56821272 |
| chr5_71507163_71507164 | chr19_5334979_5335261 | chr17_19250190_19250191 |
| chr9_131789079_131789190 | chr20_32290811_32290812 | chr8_1020857_1020858 |
| chr1_99732472_99732473 | chr1_233390249_233390250 | chr2_40470864_40470865 |
| chr6_166967377_166967378 | chr5_76429218_76429219 | chr6_21846231_21846232 |

Any method for analyzing a DNA methylation pattern may be used. For example, in embodiments, analyzing the methylation pattern includes bisulfite sequencing.

In embodiments, the DNA is sequenced using long read sequencing.

In embodiments, the subject has no symptoms of a neurodegenerative disease or condition. In embodiments, the subject has symptoms of a neurodegenerative disease or condition. In embodiments, the subject has mild symptoms of a neurodegenerative disease or condition. In embodiments, the subject has early symptoms of a neurodegenerative disease or condition. In embodiments, the subject has mild cognitive impairment.

In embodiments, the methods described herein may be used for pre-symptomatic diagnosis of a neurodegenerative disease. For example, a subject may be tested at a clinical appointment, e.g. annual physicals for ages 45-70.

In embodiments, the method includes selecting a subject having symptoms of a neurodegenerative disease or condition. In embodiments, the method includes selecting a subject having mild symptoms of a neurodegenerative disease or condition. In embodiments, the method includes selecting a subject having early symptoms of a neurodegenerative disease or condition. In embodiments, the method includes selecting a subject having mild cognitive impairment.

In an aspect is provided a method of treating a subject having a mild traumatic brain injury, including:
(i) selecting a subject at risk for mild traumatic brain injury;
(ii) obtaining cell-free DNA from a blood sample from the subject;
(iii) analyzing a methylation pattern of the cell-free DNA;
(iv) determining a percentage of cell-free DNA from neurons; and
(v) comparing the percentage of cell-free DNA from neurons to a control;
(vi) treating the subject for mild traumatic brain injury when the percentage of cell-free DNA from neurons is greater than the control.

In embodiments, treating the subject includes administering a therapeutic agent that treats one or more symptoms of mild traumatic brain injury. In embodiments, treating the subject includes physical and/or mental rest. In embodiments, treating the subject includes discontinuing one or more activities, such as sports.

In embodiments, the method further includes repeating steps (ii) to (v) at a timepoint after treatment. In embodiments, treatment is discontinued when the percentage of cell-free DNA from neurons at the timepoint is less than or equal to the control. In embodiments, treatment is continued when the percentage of cell-free DNA from neurons at the timepoint is greater than the control.

In an aspect is provided a method of monitoring a subject having or suspected of having a mild traumatic brain injury, including:
(i) selecting a subject at risk for mild traumatic brain injury;
(ii) obtaining cell-free DNA from a blood sample from the subject;
(iii) analyzing a methylation pattern of the cell-free DNA;
(iv) determining a percentage of cell-free DNA from neurons.

In embodiments, the method includes comparing the percentage of cell-free DNA from neurons to a control. In embodiments, the subject is determined to have mild traumatic brain injury when the percentage of cell-free DNA from neurons is greater than the control.

In embodiments, the subject is monitored over time. In embodiments, the method includes repeating steps (ii) to (iv) at least one time. In embodiments, the method includes monitoring the subject until the percentage of cell-free DNA from neurons is less than or equal to a control.

In embodiments, a percentage of cell-free DNA from neurons that is less than or equal to a control indicates the mTBI has resolved. In embodiments, the control is an initial percentage of cell-free DNA from neurons in the subject (e.g., from the initial determination step). In embodiments, a percentage of cell-free DNA from neurons that is less than or equal to a control indicates the subject's brain has recovered from the mTBI. In embodiments, a percentage of cell-free DNA from neurons that is less than or equal to a control indicates a reduction in brain swelling.

In embodiments, steps (ii) to (iv) are repeated daily. In embodiments, steps (ii) to (iv) are repeated every 2 days. In embodiments, steps (ii) to (iv) are repeated every 3 days. In embodiments, steps (ii) to (iv) are repeated every 4 days. In embodiments, steps (ii) to (iv) are repeated every 5 days. In embodiments, steps (ii) to (iv) are repeated every 6 days. In embodiments, steps (ii) to (iv) are repeated weekly. In embodiments, steps (ii) to (iv) are repeated biweekly. In embodiments, steps (ii) to (iv) are repeated at least once a week. In embodiments, steps (ii) to (iv) are repeated at least twice a week. In embodiments, steps (ii) to (iv) are repeated at least three times a week. In embodiments, steps (ii) to (iv) are repeated at least four times a week. In embodiments, steps (ii) to (iv) are repeated at least five times a week. In embodiments, steps (ii) to (iv) are repeated at least six times a week.

### Additional Methods

The methods, compositions and kits described herein may be used to monitor effectiveness of a therapeutic agent or potential therapeutic agent. For example, the effectiveness of a potential therapeutic agent may be monitored during a clinical trial. In another example, the effectiveness of a therapeutic agent may be monitored in a subject.

There are currently over 176 drugs in clinical trials for neurodegenerative diseases. Without being bound by theory, it is believed that monitoring drug effectiveness by monitoring current neuron DNA levels in the blood may allow trials to be significantly sped up. Tests will may be performed periodically (e.g., weekly, monthly), to assess drug effectiveness over time in reducing neuron DNA levels in the blood.

In some aspects, this disclosure relates to a method for determining efficacy of a potential treatment of a neurodegenerative disease or condition, the method comprising:
(i) obtaining cell-free DNA from a blood sample from a plurality of subjects, wherein the subjects have been administered the potential treatment;
(ii) analyzing a methylation pattern of the cell-free DNA for each subject;
(iii) determining a percentage of cell-free DNA from neurons for each subject; and
(iv) comparing the percentage of cell-free DNA from neurons for each subject to a control;
   wherein the potential treatment is efficacious when the percentage of cell-free DNA from neurons is less than the control.

In embodiments, a mean percentage of cell-free DNA from neurons is determined by averaging the percentage of cell-free DNA from neurons for each subject. In embodiments, the mean percentage of cell-free DNA from neurons is compared to the control. In embodiments, the potential treatment is efficacious when the mean percentage of cell-free DNA from neurons is less than the control.

In some aspects, this disclosure relates to a method for determining efficacy of a treatment of a neurodegenerative disease or condition, the method comprising:
(i) obtaining cell-free DNA from a blood sample from a subject, wherein the subject have been administered the treatment;
(ii) analyzing a methylation pattern of the cell-free DNA;
(iii) determining a percentage of cell-free DNA from neurons; and
(iv) comparing the percentage of cell-free DNA from neurons to a control;
   wherein the treatment is efficacious when the percentage of cell-free DNA from neurons is less than the control.

In embodiments, steps (ii) to (iv) are repeated daily. In embodiments, steps (ii) to (iv) are repeated every 2 days. In embodiments, steps (ii) to (iv) are repeated every 3 days. In embodiments, steps (ii) to (iv) are repeated every 4 days. In embodiments, steps (ii) to (iv) are repeated every 5 days. In embodiments, steps (ii) to (iv) are repeated every 6 days. In embodiments, steps (ii) to (iv) are repeated weekly. In embodiments, steps (ii) to (iv) are repeated biweekly. In embodiments, steps (ii) to (iv) are repeated at least once a week. In embodiments, steps (ii) to (iv) are repeated at least twice a week. In embodiments, steps (ii) to (iv) are repeated at least three times a week. In embodiments, steps (ii) to (iv) are repeated at least four times a week. In embodiments, steps (ii) to (iv) are repeated at least five times a week. In embodiments, steps (ii) to (iv) are repeated at least six times a week. In embodiments, steps (ii) to (iv) are repeated at least once a month. In embodiments, steps (ii) to (iv) are repeated at least twice a month. In embodiments, steps (ii) to (iv) are repeated at least three times a month. In embodiments, steps (ii) to (iv) are repeated at least four times a month. In embodiments, steps (ii) to (iv) are repeated at least five times a month. In embodiments, steps (ii) to (iv) are repeated at least six times a month. In embodiments, steps (ii) to (iv) are repeated at least seven times a month. In embodiments, steps (ii) to (iv) are repeated at least eight times a month.

The methods, compositions and kits described herein may be used to validate healthy control samples. Many institutions and firms purchase control samples from tissue banks for their research or clinical trials. Statistically 1 in 9 of these controls has undetected pre-symptomatic neurodegenerative disease. Control samples can be tested for pre-symptomatic disease to confirm that are in fact healthy controls.

In some aspects, this disclosure relates to a method for validating a control sample, the method comprising:
(i) obtaining cell-free DNA from the control sample or from the subject from whom the control sample was taken;
(ii) analyzing a methylation pattern of the cell-free DNA;
(iii) determining a percentage of cell-free DNA from neurons.

In embodiments, the method further includes comparing the percentage of cell-free DNA from neurons to a control. In embodiments, the control sample is validated when the percentage of cell-free DNA from neurons is less than the control.

The control may be any suitable control. In embodiments, the control is a percentage of cell-free DNA from neurons in a blood sample from an untreated (or placebo treated) subject or plurality of subjects. In embodiments, the control is a percentage of cell-free DNA from neurons in a blood sample from a healthy subject or plurality of healthy subjects. In embodiments, the control is a blood sample from the subject(s) prior to treatment. In embodiments, the control is a threshold. In embodiments, the threshold is a percentage of cell-free DNA from neurons greater than about 3%. In embodiments, the threshold is a percentage of cell-free DNA from neurons greater than about 4%. In embodiments, the threshold is a percentage of cell-free DNA from neurons greater than about 5%. In embodiments, the threshold is a percentage of cell-free DNA from neurons greater than about 6%. In embodiments, the threshold is a percentage of cell-free DNA from neurons greater than about 7%. In embodiments, the threshold is a percentage of cell-free DNA from neurons greater than about 8%. In embodiments, the threshold is a percentage of cell-free DNA from neurons greater than about 9%. In embodiments, the threshold is a percentage of cell-free DNA from neurons greater than about 10%. In embodiments, the threshold is a percentage of cell-free DNA from neurons greater than about 11%. In embodiments, the threshold is a percentage of cell-free DNA from neurons greater than about 12%. In embodiments, the threshold is a percentage of cell-free DNA from neurons greater than about 13%. In embodiments, the threshold is a percentage of cell-free DNA from neurons greater than about 14%. In embodiments, the threshold is a percentage of cell-free DNA from neurons greater than about 15%. In embodiments, the threshold is a percentage of cell-free DNA from neurons greater than about 20%.

### Parkinson's Disease

In embodiments, the methods described herein can be used to determine whether a subject has Parkinson's disease. In embodiments, the methods described herein can be used to determine whether a subject is at risk for Parkinson's disease. In embodiments, the methods described herein can be used to monitor effectiveness of a treatment or potential treatment for Parkinson's disease.

Parkinson's disease (PD) is one of the most common chronic progressive neurodegenerative disorders in older adults. The incidence of PD is reported as 1%-2% of individuals ages 65 years and older worldwide. The disease also affects a large number of younger people. Patients with Parkinson's disease suffer from impairment of motor functions such as bradykinesia, rest tremor, rigidity, postural disturbances, and gait alterations, including freezing of gait (FOG) and frequent falls. In addition to the motor functions, patients often suffer from impairment of non-motor functions such as cognitive impairment, sleep disturbances and depression. The most prominent signs and symptoms of Parkinson's disease occur when nerve cells in the basal ganglia, an area of the brain that controls movement, become impaired and/or die. Motor symptoms of Parkinson's disease (PD) are caused by the death of dopaminergic neurons in the substantia nigra pars compacta (SNc).

Currently, levodopa (e.g., INBRIJA) is the primary treatment for Parkinson's disease. Levodopa may be taken with carbidopa (e.g., SINEMET, SINEMET-CR, PARCOPA, RYTARY), which prevents or reduces some of the side effects of levodopa therapy (e.g., nausea, vomiting, low blood pressure, and restlessness) and reduces the amount of levodopa needed to improve symptoms. Other therapeutic agents that may be prescribed to treat Parkinson's symptoms include: dopamine agonists (e.g., pramipexole (MIRAPEX, MIRAPEX ER), rotigotine (NEUPRO, ELDEPRYL), apomorphine (APOKYN, KYNMOBI), ropinirole (REQUIP), piribedil, bromocriptine, abergoline, lisuride, pergolide); enzyme inhibitors (e.g., MAO-B inhibitors (e.g., selegiline (ZELAPAR), rasagiline (AZILECT), safinamide (XADAGO)); COMT inhibitors (e.g., entacapone (COMTAN), opicapone (ONGENTYS), tolcapone (TASMAR))) to increase the amount of dopamine by slowing down the enzymes that break down dopamine in the brain; amantadine (SYMMETREL, GOCOVRI, OSMOLEX) to help reduce involuntary movements; anticholinergic drugs (e.g., benztropine (COGENTIN), trihexyphenidyl (ARTANE)) to reduce tremors and muscle rigidity; adenosine receptor antagonists (A2A receptor antagonist) (e.g., istradefylline (NOURIANZ); and pimavanserin (NUPLAZID). Other combination treatments include carbidopa-levodopa-entacapone (DUOPA, STALEVO).

In embodiments, a subject with an increased amount of cfDNA from neurons is administered one or more therapeutic agents for treatment of Parkinson's Disease. In embodiments, the subject is administered levodopa. In embodiments, the subject is administered carbidopa. In embodiments, the subject is administered a dopamine agonist. In embodiments, the subject is administered pramipexole. In embodiments, the subject is administered rotigotine. In embodiments, the subject is administered apomorphine. In embodiments, the subject is administered ropinirole. In embodiments, the subject is administered piribedil. In embodiments, the subject is administered bromocriptine. In embodiments, the subject is administered abergoline. In embodiments, the subject is administered lisuride. In embodiments, the subject is administered pergolide. In embodiments, the subject is administered an enzyme inhibitor. In embodiments, the subject is administered an MAO-B inhibitors. In embodiments, the subject is administered selegiline. In embodiments, the subject is administered rasagiline. In embodiments, the subject is administered safinamide. In embodiments, the subject is administered a OMT inhibitor. In embodiments, the subject is administered entacapone. In embodiments, the subject is administered opicapone. In embodiments, the subject is administered tolcapone. In embodiments, the subject is administered amantadine. In embodiments, the subject is administered an anticholinergic drug. In embodiments, the subject is administered benztropine. In embodiments, the subject is administered trihexyphenidyl. In embodiments, the subject is administered an adenosine receptor antagonist (A2A receptor antagonist). In embodiments, the subject is administered istradefylline. In embodiments, the subject is administered pimavanserin. In embodiments, the subject is administered carbidopa and levodopa. In embodiments, the subject is administered carbidopa, levodopa and entacapone. In embodiments, one or more agent is expressly excluded.

### Alzheimer's Disease

In embodiments, the methods described herein can be used to determine whether a subject has Alzheimer's disease. In embodiments, the methods described herein can be used to determine whether a subject is at risk for Alzheimer's disease. In embodiments, the methods described herein can be used to monitor effectiveness of a treatment or potential treatment for Alzheimer's disease.

Alzheimer's disease is a chronic neurodegenerative disease that destroys brain cells, causing brain function to deteriorate over time. Common symptoms of Alzheimer's disease include memory loss, language problems, and impulsive or unpredictable behavior. A main feature of the disease is the presence of plaques and tangles in the brain, as well as a loss of connection between neurons in the brain. Alzheimer's disease accounts for around 60-80% of cases of dementia in the United States.

While there are no known cures for Alzheimer's disease, various medications may be prescribed to reduce or slow progression of cognitive symptoms. Several cholinesterase inhibitors have been approved by the FDA for this purpose, including donepezil (ARICEPT), galantamine (RAZADYNE), rivastigmine (EXELON). Other treatments include memantine (NAMENDA), aducanumab, and lecanemab (lecanemab-irmb; LEQEMBI).

In embodiments, the subject is administered a cholinesterase inhibitor. In embodiments, the subject is administered donepezil. In embodiments, the subject is administered galantamine. In embodiments, the subject is administered rivastigmine. In embodiments, the subject is administered memantine. In embodiments, the subject is administered aducanumab. In embodiments, the subject is administered lecanemab.

Additional therapeutic agents are in clinical trials for treatment of Alzheimer's disease. These include, without limitation: Aducanumab; AGB101 (low-dose levetiracetam); Atuzaginstat (COR388); AVP-786; AXS-05; Blarcamesine (ANAVEX2-73); BPDO-1603; Brexpiprazole; Caffeine; Donanemab; Donanemab and Aducanumab; Donepezil; Escitalopram; Gantenerumab; Gantenerumab and Solanezumab; Guanfacine; GV-971; Hydralazine; Icosapent ethyl (IPE); Losartan, Amlodipine and Atorvastatin; Metformin; Nabilone; NE3107; Nilotinib BE; Octohydroaminoacridine Succinate; Omega-3 (DHA+EPA); Semaglutide; Simufilam (PTI-125); Solanezumab; Tricaprilin; TRx0237; Valiltramiprosate (ALZ-801). ABvac40; ACI-35; AD-35; AL002; Allopregnanolone; APH-1105; Baricitinib; Bepranemab; BCG vaccine; BPN14770; Bromocriptine; Bryostatin 1; BXCL-501; Canakinumab; CORT108297; Crenezumab; CST-2032; Curcumin; CY6463; DAOIB; Dapagliflozin; Daratumumab; Dasatinib and Quercetin; Deferiprone; DHA; Dronabinol; E2814; Edonerpic (T-817MA); Elayta (CT1812); ExPlas (exercised plasma); Fosgonimeton (ATH-1017); Brain Shuttle Gantenerumab (RO7126209); GB301; Grapeseed extract; GV1001; Intranasal insulin; Intranasal insulin and Empagliflozin; IONIS MAPTRx (BIIB080); JNJ-63733657; Lamivudine (3TC); Lenalidomide; Levetiracetam; L-Serine; Lupron (leuprolide acetate depot); LY3372689; Memantine; Metabolic cofactor supplementation; MIB-626; Montelukast; MW150; Neflamapimod (VX-745); Nicotinamide; Nicotine transdermal patch; Obicetrapib; Omega-3 PUFA; Pepinemab (VX15); Posiphen; Prazosin; PU-AD; Rapamycin (sirolimus); Sargramostim; Semorinemab (RO7105705); Senicapoc; Sovateltide (PMZ-1620); Simufilam (PTI-125); Suvorexant; T3D-959; TB006; Telmisartan and perindopril; Tdap vaccine; THC-free CBD oil; Thiethylperazine (TEP); Troriluzole (BHV4157); Valacyclovir; Varoglutamstat (PQ912); VGH-AD1; Xanamem; Yangxue Qingnao pills; AAV-Htert; ACU193; ASN51; BEY2153; BMS-984923; BDPP (bioactive dietary polyphenol preparation); Contraloid acetate; COR588; Dabigatran; Edicotinib (JNJ-40346527); Efavirenz; Emtricitabine; IGC AD1; Lu AF87908; LX1001; LY3372993; MK-1942 and donepezil; NNI-362; REM0046127; Salsalate; SHR-1707; TB006; Telmisartan; Trehalose; Tricaprilin (AC-1202); Vorinostat; VT301; XPro1595. Additional treatments being tested include: Allogeneic human MSCs; SNK01 (autologous natural killer cell); Allogenic adipose MSC-Exosomes; CB-AC-02 (placenta derived MSCs); Human umbilical cord blood-derived MSCs (NEUROSTEM); Allogeneic human MSCs; AstroStem (autologous adipose-derived MSCs). In embodiments, the subject is administered one or more listed agent. In embodiments, one or more agent is expressly excluded.

### Huntington's Disease

In embodiments, the methods described herein can be used to determine whether a subject has Huntington's disease. In embodiments, the methods described herein can be used to determine whether a subject is at risk for Huntington's disease. In embodiments, the methods described herein can be used to monitor effectiveness of a treatment or potential treatment for Huntington's disease.

Huntington's disease is an inherited disease that causes progressive degeneration of nerve cells in the brain. Huntington's disease usually results in movement, thinking (cognitive) and psychiatric disorders.

There are currently no treatments to stop or slow progression of Huntington's disease. Patients may by prescribed treatments to lessen the effects, including antidepressants (e.g., selective serotonin reuptake inhibitor), antipsychotic drugs, neuroleptic agents (olanzapine, tetrabenazine, aripiprazole), exercise therapy, and the like. Experimental treatments include, without limitation, valbenazine, deutetrabenazine, bevantolol hydrochloride, pridopidine, tominersen, WVE-003, ANX-005.

In embodiments, the subject is administered an antidepressant. In embodiments, the subject is administered an antipsychotic drug. In embodiments, the subject is administered a neuroleptic agent. In embodiments, the subject is administered olanzapine. In embodiments, the subject is administered tetrabenazine. In embodiments, the subject is administered aripiprazole. In embodiments, the subject is administered exercise therapy. E In embodiments, the subject is administered valbenazine. In embodiments, the subject is administered deutetrabenazine. In embodiments, the subject is administered bevantolol hydrochloride. In embodiments, the subject is administered pridopidine. In embodiments, the subject is administered tominersen. In embodiments, the subject is administered WVE-003. In embodiments, the subject is administered ANX-005.

### Amyotrophic Lateral Sclerosis (ALS)

In embodiments, the methods described herein can be used to determine whether a subject has ALS. In embodiments, the methods described herein can be used to determine whether a subject is at risk for ALS. In embodiments, the methods described herein can be used to monitor effectiveness of a treatment or potential treatment for ALS.

ALS is a progressive nervous system disease that affects nerve cells in the brain and spinal cord, causing loss of muscle control. Symptoms may include: difficulty walking or doing normal daily activities, tripping and falling, weakness in legs, feet or ankles, hand weakness or clumsiness, slurred speech or trouble swallowing, muscle cramps and twitching in arms, shoulders and tongue, inappropriate crying, laughing or yawning, and/or cognitive and behavioral changes.

Approved medications for treating ALS include riluzole (RILUTEK, EXSERVAN, TIGLUTIK kit), edaravone (RADICAVA), and sodium phenylbutyrate and taurursodiol (RELYVRIO). Dextromethorphan HBr and quinidine sulfate (NUEDEXTA) may be prescribed for treatment of pseudobulbar affect (PBA), which is characterized by frequent, involuntary, and often sudden episodes of crying and/or laughing that are exaggerated and/or don't match how you feel.

In embodiments, the subject is administered riluzole. In embodiments, the subject is administered edaravone. In embodiments, the subject is administered sodium phenylbutyrate and taurursodiol. In embodiments, the subject is administered Dextromethorphan. In embodiments, the subject is administered quinidine sulfate.

### Ataxia

In embodiments, the methods described herein can be used to determine whether a subject has ataxia. In embodiments, the methods described herein can be used to determine whether a subject is at risk for ataxia. In embodiments, the methods described herein can be used to monitor effectiveness of a treatment or potential treatment for ataxia.

Ataxia is an abnormal lack of coordination that can cause a stumbling gait, difficulty with fine motor activities, and vision and sometimes speech problems. Ataxia may be a symptom of another health problem, such as a nutritional deficit or genetic disorder.

### Multiple Sclerosis (MS)

In embodiments, the methods described herein can be used to determine whether a subject has MS. In embodiments, the methods described herein can be used to determine whether a subject is at risk for MS. In embodiments, the methods described herein can be used to monitor effectiveness of a treatment or potential treatment for MS.

MS is caused when the immune system attacks the myelin sheath protecting nerve fibers. Eventually, the disease can cause permanent damage or deterioration of the nerve fibers. Treatments include corticosteroids (e.g., glucocorticoids such as prednisone, methylprednisolone), adrenocorticotropic hormone (ACTH), plasmapheresis, interferon beta medications (AVONEX®, REBIF® (interferon beta-1a), BETASERON®, EXTAVIA® (interferon beta-1b), PLEGRIDY® (pegylated interferonbeta-1a)), glatiramer acetate (COPAXONE, GLATOPA), monoclonal antibodies (Ofatumumab (KESIMPTA, ARZERRA), rituximab, alemtuzumab (LEMTRADA®), ocrelizumab (OCREVUS), natalizumab (TYSABRI)), teriflunomide (AUBAGIO), monomethyl fumarate (BAFIERTAM™), dimethyl fumarate (TECFIDERA), diroximel fumarate (VUMERITY), fingolimod (GILENYA), cladribine (MAVENCLAD), siponimod (MAYZENT), ponesimod (PONVORY), ozanimod (ZEPOSIA), mitoxantrone.

In embodiments, the subject is administered a corticosteroid. In embodiments, the corticosteroid is a glucocorticoid. In embodiments, the subject is administered prednisone. In embodiments, the subject is administered methylprednisolone. In embodiments, the subject is administered ACTH. In embodiments, the subject is administered plasmapheresis. In embodiments, the subject is administered interferon beta medication. In embodiments, the subject is administered interferon beta-1a. In embodiments, the subject is administered interferon beta-1b. In embodiments, the subject is administered pegylated interferonbeta-1a. In embodiments, the subject is administered glatiramer acetate. In embodiments, the subject is administered a monoclonal antibody. In embodiments, the subject is administered Ofatumumab. In embodiments, the subject is administered rituximab. In embodiments, the subject is administered alemtuzumab. In embodiments, the subject is administered ocrelizumab. In embodiments, the subject is administered natalizumab. In embodiments, the subject is administered teriflunomide. In embodiments, the subject is administered monomethyl fumarate. In embodiments, the subject is administered dimethyl fumarate. In embodiments, the subject is administered diroximel fumarate. In embodiments, the subject is administered fingolimod. In embodiments, the subject is administered cladribine. In embodiments, the subject is administered siponimod. In embodiments, the subject is administered ponesimod. In embodiments, the subject is administered ozanimod. In embodiments, the subject is administered mitoxantrone.

### Multiple System Atrophy (MSA)

In embodiments, the methods described herein can be used to determine whether a subject has MSA. In embodiments, the methods described herein can be used to determine whether a subject is at risk for MSA. In embodiments, the methods described herein can be used to monitor effectiveness of a treatment or potential treatment for MSA.

Multiple system atrophy (MSA) is a rare, degenerative neurological disorder affecting autonomic functions (e.g., blood pressure) and motor control. There are two types of MSA: parkinsonian and cerebellar, depending on symptoms. Parkinsonian type symptoms may include stiff muscles, difficulty bending limbs, bradykinesia, tremors, soft voice, and balance and posture problems. Cerebellar type symptoms may include ataxia, impaired movement and coordination, dysarthia, visual disturbances, dysphagia, and changes in speech.

There is no known cure for MSA. Symptoms may be treated with medications to raise blood pressure and medications to reduce Parkinson's-like symptoms (see Parkinson's disease treatments).

### Mild Traumatic Brain Injury and Other Brain Injury

In embodiments, the methods described herein can be used to determine whether a subject has mTBI. In embodiments, the methods described herein can be used to determine the severity of mTBI. In embodiments, the methods described herein can be used to monitor a subject's recovery from mTBI. In embodiments, the methods described herein can be used to monitor effectiveness of a treatment or potential treatment for mTBI.

In embodiments, the methods described herein can be used to determine whether a subject has a brain injury. In embodiments, the methods described herein can be used to determine the severity of brain injury. In embodiments, the methods described herein can be used to monitor a subject's recovery from a brain injury. In embodiments, the methods described herein can be used to monitor effectiveness of a treatment or potential treatment for brain injury. In embodiments, the brain injury includes swelling of the brain. In embodiments, a reduction in cfDNA from neurons indicates a reduction in swelling of the brain.

Mild traumatic brain injuries (mTBI), typically including concussions, describe an insult to the brain that, in turn, can cause long term damage/injury to the brain. It most often occurs from direct contact to the head, but can also result from indirect injury (e.g., whiplash injury or violent shaking of the head). Individuals who have suffered one brain injury are more at risk for a second brain injury and, then again, are even more susceptible for subsequent injuries. The damage from successive mTBIs is recognized to be cumulative.

The long-term damage arising from mTBI include cognitive and motor skill deterioration such as psychomotor slowing, poor concentration and attention retrieval resulting in increased variability of performance, and overall executive dysfunction, as well as sleep dysfunction, and emotional/behavioral changes. Common examples of long-term effects of mTBI are found in soldiers, boxers, football players, soccer players, and the like. There are well-documented examples of individuals who, long after the occurrence of the mTBI(s), begin to manifest the cumulative damage to the brain by loss of one or more cognitive skills and/or motor skills.

There is no approved therapeutic agent for treating mild traumatic brain injury (e.g., concussion). Generally, patients are prescribed physical and mental rest, and may be prescribed pain medication (e.g., anti-inflammatory pain medication). Ghrelin is currently in clinical trials for treatment of concussion. In embodiments, the subject is administered pain medication. In embodiments, the subject is administered anti-inflammatory pain medication. In embodiments, the subject is administered ghrelin.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

One skilled in the art would understand that the examples described herein are for the sole purpose of illustration, and that the present disclosure is not limited by this illustration.

### Example 1. Analysis of Methylation of cfDNA

cfDNA was extracted from blood plasma from a human patient using the QIAamp MinElute ccfDNA Mini Kit (Cat. No. 55204) per the manufacturer's instructions, with some modifications. Briefly, magnetic beads were warmed to 37 degrees Celsius before use. Plasma was incubated with Proteinase K, magnetic beads, and buffer for about 20 min with shaking, then spun twice (30 s at 200×g per spin) and the supernatant removed. Beads were resuspended in elution buffer and incubated for 10 min with shaking at room temperature. Supernatant was removed, buffer added, and the mixture run through a MinElute column and cfDNA eluted from the column.

The cfDNA was then subjected to bisulfite conversion (EZ DNA Methylation-Lightning Kit, Zymo Research) or enzymatic conversion (NEBNext Methyl-Seq Conversion Module, New England Biolabs) to convert methylated bases.

Following Bisulfite Conversion, the region of interest was amplified. Breiefly, bisulfite converted cfDNA (2 μl), forward primer, reverse primer, and ZymoTaq Premix (25 μl, Zymo Research), and RNAse/DNAse free water were combined and run on a thermocycler under the following conditions:
1 Cycle of 95° C. for 10 minutes;
38 cycles of: 95° C. for 30 seconds, 55° C. for 40 seconds, 72° C. for 40 seconds;
1 cycle of 72° C. for 7 minutes, followed by incubation at 4° C.

Following amplification, samples were used to prepare a library followed by sequencing (Nanopore SQK-NBD114.96).

For Alzheimer's disease, amplification of chr3:42190679,42191148 was targeted. This region was selected because it differed significantly in methylation between purified neurons and blood plasma. The average signal for blood plasma was a methylation beta value of: 0.9. The average signal for Purified Neuron DNA was a methylation beta value of: .04. These values mean that across a neuron molecule at this region it will be completely unmethylated.

The actual sequence of region is: nucleotide the CTGACGTCACCCTCTAGGCGTCTGGATAGGACGATCCTGGCTACTCCCATTCAGG GCTGCTGTCCAGTGCTGCTTTATTGGCAGTGCTGCCAGGGTCTCCGTTAGCTCTCT GCAAATTGCCTTCCTTTCTGCTCCTCCTACTCCCTCCTTCCCCCATAGAATTTTTCT TTTCATTGCCCACTTTACTGTTTTGGCTCCAGACTGTCGTTAAGAATGTACAGCCT AATTCTGGTGTGTTTCGGGATATTCTTCTGTCCAGTATTCTGGAAGGGCGGGGAG GCATGGCAGCGTTTTACTTGACGTTGATGGTGCTGTGAAGTCCATTCTTTCCTCTG CAAGACTACTGACTATGCAGAAATTTATCGAAGCGGATTATTATGAACTAGACTG GTATTATGAAGAATGCTCGGATGGTAATTATGGCCCCTGCAAAACAGAGCCGGG ATGTATAGGGGTATTGTCTCCTTCTG (chr3:42190679,42191148) (SEQ ID NO:1). The bisulfite converted form of the region is: TTGACGTTATTTTTTAGGCGTTTGGATAGGACGATTTTGGTTATTTTTATTTAGGG TTGTTGTTTAGTGTTGTTTTATTGGTAGTGTTGTTAGGGTTTTCGTTAGTTTTTTGT AAATTGTTTTTTTTTTTGTTTTTTTTATTTTTTTTTTTTTTTATAGAATTTTTTTTTTT ATTGTTTATTTTATTGTTTTGGTTTTAGATTGTCGTTAAGAATGTATAGTTTAATTT TGGTGTGTTTCGGGATATTTTTTTGTTTAGTATTTTGGAAGGGCGGGGAGGTATG GTAGCGTTTTATTTGACGTTGATGGTGTTGTGAAGTTTATTTTTTTTTTTGTAAGAT TATTGATTATGTAGAAATTTATCGAAGCGGATTATTATGAATTAGATTGGTATTA TGAAGAATGTTCGGATGGTAATTATGGTTTTTGTAAAATAGAGTCGGGATGTATA GGGGTATTGTTTTTTTTTG (chr3:42190679,42191148) (SEQ ID NO:2). These are amplified using TTTTATTGTTTTGGTTTTAGATTGT (chr3:42190859,42190884) (SEQ ID NO: 3) and GTTGATGGTGTTGTGAAGTTTATTT (chr3:42190979,42191004) (SEQ ID NO: 4) as forward primers, and AAATAAACTTCACAACACCATCAAC (chr3:42190859,42190884) (SEQ ID NO:5) and ACAATCTAAAACCAAAACAATAAAA (chr3:42190979,42191004) (SEQ ID NO:6) as reverse primers.

### Embodiments

Embodiment 1. A method of treatment of a subject, wherein the subject has or is at risk of having a neurodegenerative condition, the method comprising:
(i) obtaining cell-free DNA from a blood sample from the subject;
(ii) analyzing a methylation pattern of the cell-free DNA;
(iii) determining a percentage of cell-free DNA from neurons;
(iv) comparing the percentage of cell-free DNA from neurons to a control; and
(v) administering a therapeutic agent that treats or prevents the neurodegenerative condition when the percentage of cell-free DNA from neurons is greater than the control.

Embodiment 2. The method of embodiment 1, wherein step (ii) comprises analyzing a methylation pattern of whole amplicons of DNA.

Embodiment 3. The method of embodiment 2, wherein the whole amplicons are at least about 50 base pairs (bp) in length.

Embodiment 4. The method of embodiment 3, wherein the whole amplicons are between about 50 and about 500 base pairs in length.

Embodiment 5. The method of any one of embodiments 1 to 4, wherein the therapeutic agent is administered when the percentage of cell-free DNA from neurons is greater than about 5%.

Embodiment 6. The method of any one of embodiments 1 to 4, wherein the therapeutic agent is administered when the percentage of cell-free DNA from neurons is greater than about 7%.

Embodiment 7. The method of any one of embodiments 1 to 4, wherein the therapeutic agent is administered when the percentage of cell-free DNA from neurons is greater than about 9%.

Embodiment 8. A method of treatment of a subject, wherein the subject has or is at risk of having a neurodegenerative condition, the method comprising:
(i) obtaining cell-free DNA from a blood sample from the subject;
(ii) analyzing a methylation pattern of the cell-free DNA;
(iii) determining a percentage of cell-free DNA from neurons; and
(iv) administering a therapeutic agent that treats or prevents the neurodegenerative condition when the percentage of cell-free DNA from neurons is greater than about 5%.

Embodiment 9. A method of analyzing a biological sample of a subject, the method comprising:
(i) obtaining cell-free DNA from a blood sample from the subject;
(ii) analyzing a methylation pattern of the cell-free DNA;
(iii) determining a percentage of cell-free DNA from neurons; and
(iv) comparing the percentage of cell-free DNA from neurons to a control.

Embodiment 10. A method of measuring neuron cell death in a subject, the method comprising:
(i) obtaining cell-free DNA from a blood sample from the subject;
(ii) analyzing a methylation pattern of the cell-free DNA;
(iii) determining a percentage of cell-free DNA from neurons; and
(iv) comparing the percentage of cell-free DNA from neurons to a control.

Embodiment 11. A method of selecting a patient for treatment with a therapeutic agent for treatment of a neurodegenerative condition, the method comprising:
(i) obtaining cell-free DNA from a blood sample from the subject;
(ii) analyzing a methylation pattern of the cell-free DNA;
(iii) determining a percentage of cell-free DNA from neurons; and
(iv) comparing the percentage of cell-free DNA from neurons to a control;
   wherein the patient is selected for treatment when the percentage of cell-free DNA from neurons is greater than the control.

Embodiment 12. The method of any one of embodiments 8 to 11, wherein step (ii) comprises analyzing a methylation pattern of whole amplicons of DNA.

Embodiment 13. The method of embodiment 12, wherein the whole amplicons are at least about 50 base pairs (bp) in length.

Embodiment 14. The method of embodiment 13, wherein the whole amplicons are between about 50 and about 500 base pairs in length.

Embodiment 15. The method of any one of embodiments 10 to 14, wherein the patient is selected for treatment when the percentage of cell-free DNA from neurons is greater than about 5%.

Embodiment 16. The method of embodiment 15, wherein the patient is selected for treatment when the percentage of cell-free DNA from neurons is greater than about 7%.

Embodiment 17. The method of embodiment 15, wherein the patient is selected for treatment when the percentage of cell-free DNA from neurons is greater than about 9%.

Embodiment 18. The method of any one of embodiments 1 to 17, wherein a percentage of cell-free DNA from neurons greater than the control indicates an increased risk of neurodegenerative condition or traumatic brain injury.

Embodiment 19. A computer product comprising a non-transitory computer readable medium storing a plurality of instructions that when executed control a computer system to analyze a biological sample from a subject to determine the risk of neurodegenerative condition in the subject, the biological sample comprising cell-free DNA, the instructions comprising:
(i) identifying a first DNA methylation pattern that occurs in a neuron at a rate above a threshold, wherein the first DNA methylation pattern comprises methylation at one or more methylated regions and optionally comprises no methylation at one or more unmethylated regions;
(ii) analyzing a second DNA methylation pattern of the cell-free DNA; and
(iii) computing a relative abundance of the one or more methylated regions and optionally the one or more unmethylated regions in the cell-free DNA; and
(iv) determining the risk of neurodegenerative condition in the subject by comparing the relative abundance to a control.

Embodiment 20. A method for determining efficacy of a potential treatment of a neurodegenerative condition, the method comprising:
(i) obtaining cell-free DNA from a blood sample from a plurality of subjects, wherein the subjects have been administered the potential treatment;
(ii) analyzing a methylation pattern of the cell-free DNA;
(iii) determining a percentage of cell-free DNA from neurons; and
(iv) comparing the percentage of cell-free DNA from neurons to a control;
   wherein the potential treatment is efficacious when the percentage of cell-free DNA from neurons is less than the control.

Embodiment 21. The method of embodiment 20, wherein steps (i)-(iv) are repeated at least once.

Embodiment 22. The method of embodiment 21, wherein steps (i)-(iv) are repeated weekly.

Embodiment 23. The method of any one of the above embodiments, wherein determining a percentage of cell-free DNA from neurons comprises comparing the methylation pattern of the cell-free DNA to a neuronal DNA methylation pattern, wherein the neuronal DNA methylation pattern comprises methylation at one or more methylated regions and optionally comprises no methylation at one or more unmethylated regions.

Embodiment 24. A computer-implemented method of analyzing a biological sample, comprising:
(i) identifying a first DNA methylation pattern that occurs in a neuron at a rate above a threshold, wherein the first DNA methylation pattern comprises methylation at one or more methylated regions and optionally comprises no methylation at one or more unmethylated regions;
(ii) analyzing a second DNA methylation pattern of the cell-free DNA; and
(iii) computing a relative abundance of the one or more methylated regions and optionally the one or more unmethylated regions in the cell-free DNA; and
(iv) determining the risk of neurodegenerative disease in the subject by comparing the relative abundance to a control.

Embodiment 25. The method of any one of embodiments 20 to 24, wherein step (ii) comprises analyzing a methylation pattern of whole amplicons of DNA.

Embodiment 26. The method of embodiment 25, wherein the whole amplicons are at least about 50 base pairs (bp) in length.

Embodiment 27. The method of embodiment 26, wherein the whole amplicons are between about 50 and about 500 base pairs in length.

Embodiment 28. The method of any one of the above embodiments, wherein the control is a percentage of cell-free DNA from neurons in a blood sample from an untreated subject, a blood sample from the subjects prior to treatment, or a threshold.

Embodiment 29. The method of embodiment 28, wherein the threshold is a percentage of cell-free DNA from neurons greater than about 5%.

Embodiment 30. The method of embodiment 28, wherein the threshold is a percentage of cell-free DNA from neurons greater than about 7%.

Embodiment 31. The method of embodiment 28, wherein the threshold is a percentage of cell-free DNA from neurons greater than about 9%.

Embodiment 32. The method of any one of the above embodiments, wherein the neurodegenerative disease is selected from Alzheimer's disease, Huntington disease, Parkinson's disease, amyotrophic lateral sclerosis (ALS), ataxia, multiple sclerosis, multiple system atrophy, or concussion.

Embodiment 33. The method of any one of the above embodiments, wherein the neuron is a motor neuron, a sensory neuron, an interneuron, a dopaminergic neuron, cholinergic neuron, a GABAergic neuron, a glutamatergic neuron, or a cortical neuron.

Embodiment 34. The method of any one of the above embodiments, wherein the neuron is from the forebrain, midbrain, or hindbrain.

Embodiment 35. The method of any one of the above embodiments, wherein the neuron is from the frontal lobe, temporal lobe, parietal lobe, occipital lobe, cerebellum, or brain stem.

Embodiment 36. The method of any one of the above embodiments, wherein analyzing the methylation pattern comprises converting 5-methylcytosine in the cell-free DNA to a different nucleotide.

Embodiment 37. The method of embodiment 36, wherein converting comprises bisulfite conversion or enzymatic conversion.

Embodiment 38. The method of any one of the above embodiments, wherein the subject has mild cognitive impairment.

Embodiment 39. The method of any one of the above embodiments, wherein the subject is over 45 years of age.

Embodiment 40. The method of any one of the above embodiments, comprising selecting a subject over 45 years of age.

Embodiment 41. The method of embodiment 40, wherein the subject has no symptoms of the neurodegenerative condition.

Embodiment 42. The method of any one of the above embodiments, comprising selecting a subject having mild cognitive impairment.

Embodiment 43. A method of treating a subject having a mild traumatic brain injury, comprising:
(i) selecting a subject at risk for mild traumatic brain injury;
(ii) obtaining cell-free DNA from a blood sample from the subject;
(iii) analyzing a methylation pattern of the cell-free DNA;
(iv) determining a percentage of cell-free DNA from neurons; and
(v) comparing the percentage of cell-free DNA from neurons to a control;
(vi) treating the subject for mild traumatic brain injury when the percentage of cell-free DNA from neurons is greater than the control.

Embodiment 44. The method of embodiment 43, wherein treating the subject comprises administering a therapeutic agent that treats one or more symptoms of mild traumatic brain injury.

Embodiment 45. The method of embodiment 43 or 44, further comprising repeating steps (ii) to (v) at a timepoint after treatment.

Embodiment 46. The method of embodiment 45, wherein treatment is discontinued when the percentage of cell-free DNA from neurons at the timepoint is less than or equal to the control.

Embodiment 47. The method of embodiment 46, wherein treatment is continued when the percentage of cell-free DNA from neurons at the timepoint is greater than the control.

Embodiment 48. A kit comprising a first plurality of oligonucleotides, wherein each oligonucleotide in the first plurality is capable of hybridizing to a region that is preferentially methylated in a neuron cell.

Embodiment 49. The kit of embodiment 48, further comprising a second plurality of oligonucleotides, wherein each oligonucleotide in the second plurality is capable of hybridizing to a region that is preferentially unmethylated in a neuron cell.

Embodiment 50. The kit of embodiment 48 or 49 for determining efficacy of a potential treatment of a neurodegenerative disease or condition.

Embodiment 51. The kit of embodiment 50, wherein the neurodegenerative disease or condition is selected from Alzheimer's disease, Huntington disease, Parkinson's disease, amyotrophic lateral sclerosis (ALS), ataxia, multiple sclerosis, multiple system atrophy, or concussion.

Embodiment 52. The kit of any one of embodiments 48 to 51, wherein the neuron is a motor neuron, a sensory neuron, an interneuron, a dopaminergic neuron, cholinergic neuron, a GABAergic neuron, a glutamatergic neuron, or a cortical neuron.

Embodiment 53. The kit of any one of embodiments 48 to 52, wherein the neuron is from the forebrain, midbrain, or hindbrain.

Embodiment 54. The kit of any one of embodiments 48 to 53, wherein the neuron is from the frontal lobe, temporal lobe, parietal lobe, occipital lobe, cerebellum, or brain stem.

Embodiment 55. The kit of any one of embodiments 48 to 54, wherein the plurality of oligonucleotides comprises one or more oligonucleotides having a nucleotide sequence of any one of SEQ ID NOs: 3-6.

Embodiment 56. A method of detecting cell-free DNA from neurons in a blood sample, comprising:
(i) obtaining cell-free DNA from a blood sample from a human subject; and
(ii) detecting whether cell-free DNA from neurons is present in the blood sample by methylation analysis comprising subjecting the cell-free DNA from the blood sample to sequencing of whole amplicons of DNA.

Embodiment 57. The method of embodiment 56, wherein the whole amplicons are at least about 50 base pairs (bp) in length.

Embodiment 58. The method of embodiment 57, wherein the whole amplicons are between about 50 and about 500 base pairs in length.

Embodiment 59. The method of any one of embodiments 56 to 58, wherein the whole amplicons were produced using one or more primers that comprise the sequence of any one of SEQ ID NOs: 3-6.

Embodiment 60. The method of any one of embodiments 56 to 59, wherein the whole amplicons were produced using one or more primers targeting a region selected from the regions listed in Table 1.

Embodiment 61. A method for determining the methylation status of an amplicon, comprising:
(i) obtaining cell-free DNA from a blood sample from a human subject;
(ii) converting 5-methylcytosine in the cell-free DNA to a different nucleotide, thereby producing converted cell-free DNA;
(iii) amplifying the converted cell-free DNA, thereby producing an amplicon; and
(iv) sequencing the amplicon, wherein the amplicon is between about 50 bp and about 500 bp in length.

Embodiment 62. The method of embodiment 61, wherein the whole amplicons were produced using one or more primers that comprise the sequence of any one of SEQ ID NOs: 3-6.

Embodiment 63. The method of embodiment 61, wherein the whole amplicons were produced using one or more primers targeting a region selected from the regions listed in Table 1.

TABLE 2
Exemplary analysis of methylation pattern of cell-free DNA as provided herein.

| | | | | | Reads | | | | | | |
|---|---|---|---|---|---|---|---|---|---|---|---|
| | | | | | With | | Reads | | | | |
| | | | | | Primers | Reads | Identified | Reads | Ratio of | | Ratio of |
| | | | | Reads | and All | Identified | as | Identified | Blood | Ratio of | Neuron |
| Sample_{—} | | Tissue | Total | Including | Target | as Blood | Unknown | as Neuron | Derived | Unknown | Derived |
| ID | Diagnosis | Type | Reads | Primers | CG's | Derived | Derived | Derived | DNA | DNA | DNA |
| Sample_{—} | Control | Blood | 13955 | 230 | 3 | 3 | 0 | 0 | 1 | 0 | 0 |
| 1 | Pooled | Plasma | | | | | | | | | |
| Sample_{—} | Control | Blood | 18886 | 509 | 31 | 30 | 1 | 0 | 0.967741935 | 0.032258065 | 0 |
| 2 | Pooled | Plasma | | | | | | | | | |
| Sample_{—} | Control | Blood | 19845 | 332 | 14 | 13 | 1 | 0 | 0.928571429 | 0.071428571 | 0 |
| 3 | Pooled | Plasma | | | | | | | | | |
| Sample_{—} | Control | Blood | 20602 | 451 | 35 | 34 | 1 | 0 | 0.971428571 | 0.028571429 | 0 |
| 4 | Pooled | Plasma | | | | | | | | | |
| Sample_{—} | Control | Blood | 20460 | 14472 | 1812 | 1811 | 1 | 0 | 0.999448124 | 0.000551876 | 0 |
| 5 | Single | Plasma | | | | | | | | | |
| | Donor | | | | | | | | | | |
| Sample_{—} | Control | Blood | 14463 | 4560 | 114 | 111 | 3 | 0 | 0.973684211 | 0.026315789 | 0 |
| 6 | Single | Plasma | | | | | | | | | |
| | Donor | | | | | | | | | | |
| Sample_{—} | Control | Blood | 38795 | 13326 | 534 | 522 | 12 | 0 | 0.97752809 | 0.02247191 | 0 |
| 7 | Single | Plasma | | | | | | | | | |
| | Donor | | | | | | | | | | |
| Sample_{—} | Control | Blood | 71392 | 21631 | 1612 | 1597 | 15 | 0 | 0.990694789 | 0.009305211 | 0 |
| 8 | Single | Plasma | | | | | | | | | |
| | Donor | | | | | | | | | | |
| Sample_{—} | Control | Blood | 48135 | 14757 | 1218 | 1215 | 3 | 0 | 0.997536946 | 0.002463054 | 0 |
| 9 | Single | Plasma | | | | | | | | | |
| | Donor | | | | | | | | | | |
| Sample_{—} | Control | Blood | 15209 | 5386 | 68 | 68 | 0 | 0 | 1 | 0 | 0 |
| 10 | Single | Plasma | | | | | | | | | |
| | Donor | | | | | | | | | | |
| Sample_{—} | Control | Blood | 42151 | 14678 | 1111 | 1093 | 18 | 0 | 0.98379838 | 0.01620162 | 0 |
| 11 | Single | Plasma | | | | | | | | | |
| | Donor | | | | | | | | | | |
| Sample_{—} | Control | Blood | 33903 | 11956 | 1004 | 994 | 10 | 0 | 0.990039841 | 0.009960159 | 0 |
| 12 | Single | Plasma | | | | | | | | | |
| | Donor | | | | | | | | | | |
| Sample_{—} | Control | Blood | 14891 | 5547 | 395 | 395 | 0 | 0 | 1 | 0 | 0 |
| 13 | Single | Plasma | | | | | | | | | |
| | Donor | | | | | | | | | | |
| Sample_{—} | Control | Blood | 24869 | 9935 | 662 | 656 | 6 | 0 | 0.990936556 | 0.009063444 | 0 |
| 14 | Single | Plasma | | | | | | | | | |
| | Donor | | | | | | | | | | |
| Sample_{—} | Control | Blood | 28058 | 9211 | 528 | 523 | 5 | 0 | 0.990530303 | 0.009469697 | 0 |
| 15 | Single | Plasma | | | | | | | | | |
| | Donor | | | | | | | | | | |
| Sample_{—} | Control | Blood | 53911 | 16754 | 1305 | 1274 | 31 | 0 | 0.976245211 | 0.023754789 | 0 |
| 16 | Single | Plasma | | | | | | | | | |
| | Donor | | | | | | | | | | |
| Sample_{—} | Control | Blood | 27373 | 9559 | 368 | 365 | 3 | 0 | 0.991847826 | 0.008152174 | 0 |
| 17 | Single | Plasma | | | | | | | | | |
| | Donor | | | | | | | | | | |
| Sample_{—} | Control | Blood | 51398 | 18196 | 800 | 775 | 25 | 0 | 0.96875 | 0.03125 | 0 |
| 18 | Single | Plasma | | | | | | | | | |
| | Donor | | | | | | | | | | |
| Sample_{—} | Control | Blood | 28089 | 10742 | 690 | 679 | 11 | 0 | 0.984057971 | 0.015942029 | 0 |
| 19 | Single | Plasma | | | | | | | | | |
| | Donor | | | | | | | | | | |
| Sample_{—} | Control | Blood | 61490 | 18018 | 1084 | 1073 | 11 | 0 | 0.989852399 | 0.010147601 | 0 |
| 20 | Single | Plasma | | | | | | | | | |
| | Donor | | | | | | | | | | |
| Sample_{—} | Control | Blood | 62416 | 19357 | 1342 | 1331 | 11 | 0 | 0.991803279 | 0.008196721 | 0 |
| 21 | Single | Plasma | | | | | | | | | |
| | Donor | | | | | | | | | | |
| Sample_{—} | Control | Blood | 21241 | 7637 | 577 | 568 | 9 | 0 | 0.98440208 | 0.01559792 | 0 |
| 22 | Single | Plasma | | | | | | | | | |
| | Donor | | | | | | | | | | |
| Sample_{—} | Control | Blood | 22611 | 7867 | 449 | 440 | 9 | 0 | 0.979955457 | 0.020044543 | 0 |
| 23 | Single | Plasma | | | | | | | | | |
| | Donor | | | | | | | | | | |
| Sample_{—} | Control | Blood | 52579 | 17354 | 1316 | 1300 | 16 | 0 | 0.987841945 | 0.012158055 | 0 |
| 24 | Single | Plasma | | | | | | | | | |
| | Donor | | | | | | | | | | |
| Sample_{—} | Control | Blood | 72437 | 22155 | 1337 | 1313 | 24 | 0 | 0.982049364 | 0.017950636 | 0 |
| 25 | Single | Plasma | | | | | | | | | |
| | Donor | | | | | | | | | | |
| Sample_{—} | Control | Blood | 46657 | 16111 | 1105 | 1094 | 11 | 0 | 0.990045249 | 0.009954751 | 0 |
| 26 | Single | Plasma | | | | | | | | | |
| | Donor | | | | | | | | | | |
| Sample_{—} | Control | Blood | 53052 | 16144 | 1306 | 1296 | 10 | 0 | 0.992343032 | 0.007656968 | 0 |
| 27 | Single | Plasma | | | | | | | | | |
| | Donor | | | | | | | | | | |
| Sample_{—} | Control | Blood | 39434 | 13745 | 1142 | 1124 | 18 | 0 | 0.984238179 | 0.015761821 | 0 |
| 28 | Single | Plasma | | | | | | | | | |
| | Donor | | | | | | | | | | |
| Sample_{—} | MCI | Blood | 70529 | 21615 | 1816 | 1795 | 21 | 0 | 0.988436123 | 0.011563877 | 0 |
| 29 | Never | Plasma | | | | | | | | | |
| | Fully | | | | | | | | | | |
| | Progressed | | | | | | | | | | |
| | to ALZ | | | | | | | | | | |
| Sample_{—} | Control | Blood | 91348 | 26625 | 1864 | 1833 | 31 | 0 | 0.983369099 | 0.016630901 | 0 |
| 30 | Single | Plasma | | | | | | | | | |
| | Donor | | | | | | | | | | |
| Sample_{—} | Control | Blood | 23316 | 8241 | 582 | 578 | 4 | 0 | 0.993127148 | 0.006872852 | 0 |
| 31 | Single | Plasma | | | | | | | | | |
| | Donor | | | | | | | | | | |
| Sample_{—} | Control | Blood | 29635 | 11480 | 123 | 122 | 1 | 0 | 0.991869919 | 0.008130081 | 0 |
| 32 | Single | Plasma | | | | | | | | | |
| | Donor | | | | | | | | | | |
| Sample_{—} | Control | Blood | 71351 | 23768 | 240 | 233 | 7 | 0 | 0.970833333 | 0.029166667 | 0 |
| 33 | Single | Plasma | | | | | | | | | |
| | Donor | | | | | | | | | | |
| Sample_{—} | Control | Blood | 78203 | 22036 | 1571 | 1562 | 9 | 0 | 0.994271165 | 0.005728835 | 0 |
| 34 | Single | Plasma | | | | | | | | | |
| | Donor | | | | | | | | | | |
| Sample_{—} | Control | Blood | 48466 | 15990 | 1125 | 1108 | 17 | 0 | 0.984888889 | 0.015111111 | 0 |
| 35 | Single | Plasma | | | | | | | | | |
| | Donor | | | | | | | | | | |
| Sample_{—} | Control | Blood | 29221 | 10784 | 700 | 672 | 28 | 0 | 0.96 | 0.04 | 0 |
| 36 | Single | Plasma | | | | | | | | | |
| | Donor | | | | | | | | | | |
| Sample_{—} | Control | Blood | 19148 | 7490 | 415 | 410 | 5 | 0 | 0.987951807 | 0.012048193 | 0 |
| 37 | Single | Plasma | | | | | | | | | |
| | Donor | | | | | | | | | | |
| Sample_{—} | Control | Blood | 51382 | 18105 | 1331 | 1323 | 8 | 0 | 0.993989482 | 0.006010518 | 0 |
| 38 | Single | Plasma | | | | | | | | | |
| | Donor | | | | | | | | | | |
| Sample_{—} | Control | Blood | 42287 | 14606 | 1028 | 1023 | 5 | 0 | 0.995136187 | 0.004863813 | 0 |
| 39 | Single | Plasma | | | | | | | | | |
| | Donor | | | | | | | | | | |
| Sample_{—} | Control | Blood | 51081 | 17043 | 1112 | 1100 | 12 | 0 | 0.989208633 | 0.010791367 | 0 |
| 40 | Single | Plasma | | | | | | | | | |
| | Donor | | | | | | | | | | |
| Sample_{—} | Control | Blood | 16214 | 6352 | 413 | 410 | 3 | 0 | 0.992736077 | 0.007263923 | 0 |
| 41 | Single | Plasma | | | | | | | | | |
| | Donor | | | | | | | | | | |
| Sample_{—} | Control | Blood | 21661 | 7491 | 579 | 570 | 9 | 0 | 0.984455959 | 0.015544041 | 0 |
| 42 | Single | Plasma | | | | | | | | | |
| | Donor | | | | | | | | | | |
| Sample_{—} | Control | Blood | 24843 | 8555 | 588 | 585 | 3 | 0 | 0.994897959 | 0.005102041 | 0 |
| 43 | Single | Plasma | | | | | | | | | |
| | Donor | | | | | | | | | | |
| Sample_{—} | Control | Blood | 63035 | 18928 | 1240 | 1230 | 10 | 0 | 0.991935484 | 0.008064516 | 0 |
| 44 | Single | Plasma | | | | | | | | | |
| | Donor | | | | | | | | | | |
| Sample_{—} | Control | Blood | 29165 | 11887 | 29 | 29 | 0 | 0 | 1 | 0 | 0 |
| 45 | Single | Plasma | | | | | | | | | |
| | Donor | | | | | | | | | | |
| Sample_{—} | Control | Blood | 42968 | 15446 | 719 | 707 | 12 | 0 | 0.983310153 | 0.016689847 | 0 |
| 46 | Single | Plasma | | | | | | | | | |
| | Donor | | | | | | | | | | |
| Sample_{—} | MCI | Blood | 22383 | 8587 | 628 | 625 | 3 | 0 | 0.99522293 | 0.00477707 | 0 |
| 47 | Never | Plasma | | | | | | | | | |
| | Fully | | | | | | | | | | |
| | Progressed | | | | | | | | | | |
| | to ALZ | | | | | | | | | | |
| Sample_{—} | Control | Blood | 74714 | 23211 | 1741 | 1714 | 27 | 0 | 0.984491671 | 0.015508329 | 0 |
| 48 | Single | Plasma | | | | | | | | | |
| | Donor | | | | | | | | | | |
| Sample_{—} | Control | Blood | 45134 | 13924 | 1049 | 1034 | 15 | 0 | 0.985700667 | 0.014299333 | 0 |
| 49 | Single | Plasma | | | | | | | | | |
| | Donor | | | | | | | | | | |
| Sample_{—} | Control | Blood | 40145 | 13632 | 903 | 902 | 1 | 0 | 0.99889258 | 0.00110742 | 0 |
| 50 | Single | Plasma | | | | | | | | | |
| | Donor | | | | | | | | | | |
| Sample_{—} | Control | Blood | 16323 | 5869 | 378 | 372 | 6 | 0 | 0.984126984 | 0.015873016 | 0 |
| 51 | Single | Plasma | | | | | | | | | |
| | Donor | | | | | | | | | | |
| Sample_{—} | Control | Blood | 42702 | 13924 | 990 | 974 | 15 | 1 | 0.983838384 | 0.015151515 | 0.001010101 |
| 52 | Single | Plasma | | | | | | | | | |
| | Donor | | | | | | | | | | |
| Sample_{—} | MCI | Blood | 36934 | 13296 | 948 | 942 | 5 | 1 | 0.993670886 | 0.005274262 | 0.001054852 |
| 53 | Never | Plasma | | | | | | | | | |
| | Fully | | | | | | | | | | |
| | Progressed | | | | | | | | | | |
| | to ALZ | | | | | | | | | | |
| Sample_{—} | Control | Blood | 25012 | 8987 | 719 | 691 | 27 | 1 | 0.961057024 | 0.037552156 | 0.001390821 |
| 54 | Single | Plasma | | | | | | | | | |
| | Donor | | | | | | | | | | |
| Sample_{—} | Control | Blood | 44955 | 16085 | 1240 | 1229 | 7 | 4 | 0.991129032 | 0.005645161 | 0.003225806 |
| 55 | Single | Plasma | | | | | | | | | |
| | Donor | | | | | | | | | | |
| Sample_{—} | MCI | Blood | 62232 | 19895 | 1205 | 1191 | 10 | 4 | 0.988381743 | 0.008298755 | 0.003319502 |
| 56 | Never | Plasma | | | | | | | | | |
| | Fully | | | | | | | | | | |
| | Progressed | | | | | | | | | | |
| | to ALZ | | | | | | | | | | |
| Sample_{—} | Control | Blood | 54763 | 18002 | 235 | 223 | 11 | 1 | 0.94893617 | 0.046808511 | 0.004255319 |
| 57 | Single | Plasma | | | | | | | | | |
| | Donor | | | | | | | | | | |
| Sample_{—} | Control | Blood | 42523 | 13821 | 973 | 959 | 7 | 7 | 0.985611511 | 0.007194245 | 0.007194245 |
| 58 | Single | Plasma | | | | | | | | | |
| | Donor | | | | | | | | | | |
| Sample_{—} | MCI | Blood | 48964 | 16110 | 1166 | 1141 | 15 | 10 | 0.978559177 | 0.012864494 | 0.008576329 |
| 59 | Never | Plasma | | | | | | | | | |
| | Fully | | | | | | | | | | |
| | Progressed | | | | | | | | | | |
| | to ALZ | | | | | | | | | | |
| Sample_{—} | Control | Blood | 47744 | 14755 | 1030 | 997 | 23 | 10 | 0.967961165 | 0.022330097 | 0.009708738 |
| 60 | Single | Plasma | | | | | | | | | |
| | Donor | | | | | | | | | | |
| Sample_{—} | Control | Blood | 49946 | 17234 | 1290 | 1265 | 8 | 17 | 0.980620155 | 0.00620155 | 0.013178295 |
| 61 | Single | Plasma | | | | | | | | | |
| | Donor | | | | | | | | | | |
| Sample_{—} | Control | Blood | 34082 | 18179 | 2573 | 2535 | 2 | 36 | 0.985231248 | 0.000777303 | 0.01399145 |
| 62 | Single | Plasma | | | | | | | | | |
| | Donor | | | | | | | | | | |
| Sample_{—} | MCI | Blood | 40165 | 13709 | 1254 | 1024 | 9 | 221 | 0.977099237 | 0.008587786 | 0.210877863 |
| 63 | Later | Plasma | | | | | | | | | |
| | Diagnosed | | | | | | | | | | |
| | ALZ | | | | | | | | | | |
| Sample_{—} | ALZ | Blood | 42723 | 13299 | 1160 | 973 | 16 | 171 | 0.969123506 | 0.015936255 | 0.170318725 |
| 64 | | Plasma | | | | | | | | | |
| Sample_{—} | ALZ | Blood | 47765 | 14555 | 1192 | 895 | 11 | 286 | 0.970715835 | 0.011930586 | 0.310195228 |
| 65 | | Plasma | | | | | | | | | |
| Sample_{—} | MCI | Blood | 38824 | 13966 | 1164 | 981 | 28 | 155 | 0.954280156 | 0.027237354 | 0.15077821 |
| 66 | Later | Plasma | | | | | | | | | |
| | Diagnosed | | | | | | | | | | |
| | ALZ | | | | | | | | | | |
| Sample_{—} | ALZ | Blood | 64032 | 22047 | 1766 | 1618 | 29 | 119 | 0.959667853 | 0.017200474 | 0.070581257 |
| 67 | | Plasma | | | | | | | | | |
| Sample_{—} | Control | Blood | 3289 | 1947 | 307 | 283 | 6 | 18 | 0.956081081 | 0.02027027 | 0.060810811 |
| 68 | Single | Plasma | | | | | | | | | |
| | Donor | | | | | | | | | | |
| Sample_{—} | ALZ | Blood | 25045 | 8473 | 221 | 195 | 3 | 23 | 0.960591133 | 0.014778325 | 0.113300493 |
| 69 | | Plasma | | | | | | | | | |
| Sample_{—} | MCI | Blood | 3125 | 1431 | 178 | 158 | 0 | 20 | 0.969325153 | 0 | 0.122699387 |
| 70 | Never | Plasma | | | | | | | | | |
| | Fully | | | | | | | | | | |
| | Progressed | | | | | | | | | | |
| | to ALZ | | | | | | | | | | |
| Sample_{—} | ALZ | Blood | 36269 | 10859 | 774 | 670 | 12 | 92 | 0.949008499 | 0.016997167 | 0.130311615 |
| 71 | | Plasma | | | | | | | | | |
| Sample_{—} | ALZ | Blood | 28649 | 9253 | 687 | 543 | 16 | 128 | 0.937823834 | 0.027633851 | 0.221070812 |
| 72 | | Plasma | | | | | | | | | |
| Sample_{—} | ALZ | Blood | 91789 | 27565 | 731 | 576 | 9 | 146 | 0.94892916 | 0.014827018 | 0.240527183 |
| 73 | | Plasma | | | | | | | | | |
| Sample_{—} | MCI | Blood | 7311 | 4311 | 789 | 611 | 0 | 178 | 0.962204724 | 0 | 0.280314961 |
| 74 | Never | Plasma | | | | | | | | | |
| | Fully | | | | | | | | | | |
| | Progressed | | | | | | | | | | |
| | to ALZ | | | | | | | | | | |
| Sample_{—} | ALZ | Blood | 23441 | 8085 | 667 | 578 | 4 | 85 | 0.95379538 | 0.00660066 | 0.140264026 |
| 75 | | Plasma | | | | | | | | | |
| Sample_{—} | ALZ | Blood | 46158 | 14375 | 1245 | 1067 | 19 | 159 | 0.942579505 | 0.016784452 | 0.140459364 |
| 76 | | Plasma | | | | | | | | | |
| Sample_{—} | ALZ | Blood | 28099 | 9378 | 836 | 744 | 13 | 79 | 0.941772152 | 0.016455696 | 0.1 |
| 77 | | Plasma | | | | | | | | | |
| Sample_{—} | Control | Blood | 40609 | 14219 | 1185 | 951 | 12 | 222 | 0.944389275 | 0.011916584 | 0.220456802 |
| 78 | Single | Plasma | | | | | | | | | |
| | Donor | | | | | | | | | | |
| Sample_{—} | ALZ | Blood | 33914 | 10871 | 754 | 637 | 26 | 91 | 0.917867435 | 0.037463977 | 0.131123919 |
| 79 | | Plasma | | | | | | | | | |
| Sample_{—} | Control | Blood | 36212 | 13199 | 1151 | 1044 | 18 | 89 | 0.938848921 | 0.01618705 | 0.080035971 |
| 80 | Single | Plasma | | | | | | | | | |
| | Donor | | | | | | | | | | |
| Sample_{—} | ALZ | Blood | 29968 | 11009 | 828 | 707 | 15 | 106 | 0.935185185 | 0.01984127 | 0.14021164 |
| 81 | | Plasma | | | | | | | | | |
| Sample_{—} | ALZ | Blood | 16604 | 5840 | 581 | 393 | 4 | 184 | 0.940191388 | 0.009569378 | 0.440191388 |
| 82 | | Plasma | | | | | | | | | |
| Sample_{—} | ALZ | Blood | 44521 | 15021 | 890 | 694 | 22 | 174 | 0.920424403 | 0.029177719 | 0.230769231 |
| 83 | | Plasma | | | | | | | | | |
| Sample_{—} | ALZ | Blood | 19895 | 7084 | 602 | 520 | 14 | 68 | 0.920353982 | 0.024778761 | 0.120353982 |
| 84 | | Plasma | | | | | | | | | |
| Sample_{—} | MCI | Blood | 60556 | 21016 | 1543 | 1325 | 18 | 200 | 0.930477528 | 0.012640449 | 0.140449438 |
| 85 | Later | Plasma | | | | | | | | | |
| | Diagnosed | | | | | | | | | | |
| | ALZ | | | | | | | | | | |
| Sample_{—} | ALZ | Blood | 28657 | 10880 | 865 | 629 | 11 | 225 | 0.925 | 0.016176471 | 0.330882353 |
| 86 | | Plasma | | | | | | | | | |
| Sample_{—} | ALZ | Blood | 48985 | 15928 | 299 | 260 | 5 | 34 | 0.921985816 | 0.017730496 | 0.120567376 |
| 87 | | Plasma | | | | | | | | | |
| Sample_{—} | ALZ | Blood | 30836 | 9289 | 625 | 451 | 14 | 160 | 0.907444668 | 0.028169014 | 0.32193159 |
| 88 | | Plasma | | | | | | | | | |
| Sample_{—} | ALZ | Blood | 44983 | 15139 | 1066 | 890 | 26 | 150 | 0.894472362 | 0.026130653 | 0.150753769 |
| 89 | | Plasma | | | | | | | | | |
| Sample_{—} | Control | Blood | 40396 | 14320 | 548 | 478 | 15 | 55 | 0.872262774 | 0.027372263 | 0.100364964 |
| 90 | Single | Plasma | | | | | | | | | |
| | Donor | | | | | | | | | | |
| Sample_{—} | ALZ | Blood | 17263 | 6282 | 490 | 426 | 13 | 51 | 0.869387755 | 0.026530612 | 0.104081633 |
| 91 | | Plasma | | | | | | | | | |
| Sample_{—} | ALZ | Blood | 2342 | 922 | 70 | 61 | 0 | 9 | 0.871428571 | 0 | 0.128571429 |
| 92 | | Plasma | | | | | | | | | |
| Sample_{—} | MCI | Blood | 32565 | 12117 | 889 | 736 | 23 | 130 | 0.827896513 | 0.025871766 | 0.146231721 |
| 93 | Later | Plasma | | | | | | | | | |
| | Diagnosed | | | | | | | | | | |
| | ALZ | | | | | | | | | | |
| Sample_{—} | ALZ | Blood | 32434 | 11981 | 254 | 192 | 23 | 39 | 0.755905512 | 0.090551181 | 0.153543307 |
| 94 | | Plasma | | | | | | | | | |
| Sample_{—} | MCI | Blood | 365 | 217 | 31 | 23 | 3 | 5 | 0.741935484 | 0.096774194 | 0.161290323 |
| 95 | Later | Plasma | | | | | | | | | |
| | Diagnosed | | | | | | | | | | |
| | ALZ | | | | | | | | | | |
| Sample_{—} | ALZ | Blood | 125 | 63 | 7 | 5 | 0 | 2 | 0.714285714 | 0 | 0.285714286 |
| 96 | | Plasma | | | | | | | | | |
| Sample_{—} | Control | Blood | 1620 | 1053 | 73 | 49 | 0 | 24 | 0.671232877 | 0 | 0.328767123 |
| 97 | Single | Plasma | | | | | | | | | |
| | Donor | | | | | | | | | | |
| Sample_{—} | MCI | Blood | 1944 | 1236 | 81 | 47 | 1 | 33 | 0.580246914 | 0.012345679 | 0.407407407 |
| 98 | Later | Plasma | | | | | | | | | |
| | Diagnosed | | | | | | | | | | |
| | ALZ | | | | | | | | | | |
| Sample_{—} | ALZ | Blood | 1539 | 720 | 67 | 34 | 2 | 31 | 0.507462687 | 0.029850746 | 0.462686567 |
| 99 | | Plasma | | | | | | | | | |
| Sample_{—} | ALZ | Blood | 1506 | 898 | 100 | 33 | 1 | 66 | 0.33 | 0.01 | 0.66 |
| 100 | | Plasma | | | | | | | | | |
| Sample_{—} | Purified | Dopaminergic | 101221 | 4159 | 514 | 6 | 102 | 406 | 0.011673152 | 0.19844358 | 0.789883268 |
| 101 | Neuron | Neurons | | | | | | | | | |
| Sample_{—} | Purified | Dopaminergic | 120090 | 7517 | 904 | 3 | 166 | 735 | 0.003318584 | 0.183628319 | 0.813053097 |
| 102 | Neuron | Neurons | | | | | | | | | |
| Sample_{—} | Purified | Dopaminergic | 74131 | 2516 | 325 | 1 | 58 | 266 | 0.003076923 | 0.178461538 | 0.818461538 |
| 103 | Neuron | Neurons | | | | | | | | | |
| Sample_{—} | Purified | Dopaminergic | 33381 | 1215 | 109 | 1 | 17 | 91 | 0.009174312 | 0.155963303 | 0.834862385 |
| 104 | Neuron | Neurons | | | | | | | | | |

What is claimed is:

1. A method of treatment of a subject, wherein the subject has or is at risk of having a neurodegenerative condition, the method comprising:
(i) obtaining cell-free DNA from a blood sample from the subject;
(ii) analyzing a methylation pattern of the cell-free DNA;
(iii) determining a percentage of cell-free DNA from neurons;
(iv) comparing the percentage of cell-free DNA from neurons to a control; and
(v) administering a therapeutic agent that treats or prevents the neurodegenerative condition when the percentage of cell-free DNA from neurons is greater than the control.

2. The method of claim 1, wherein step (ii) comprises analyzing a methylation pattern of whole amplicons of DNA.

3. The method of claim 2, wherein the whole amplicons are at least about 50 base pairs (bp) in length.

4. The method of claim 3, wherein the whole amplicons are between about 50 and about 500 base pairs in length.

5. The method of any one of claims 1 to 4, wherein the therapeutic agent is administered when the percentage of cell-free DNA from neurons is greater than about 5%.

6. The method of any one of claims 1 to 4, wherein the therapeutic agent is administered when the percentage of cell-free DNA from neurons is greater than about 7%.

7. The method of any one of claims 1 to 4, wherein the therapeutic agent is administered when the percentage of cell-free DNA from neurons is greater than about 9%.

8. A method of treatment of a subject, wherein the subject has or is at risk of having a neurodegenerative condition, the method comprising:
(i) obtaining cell-free DNA from a blood sample from the subject;
(ii) analyzing a methylation pattern of the cell-free DNA;
(iii) determining a percentage of cell-free DNA from neurons; and
(iv) administering a therapeutic agent that treats or prevents the neurodegenerative condition when the percentage of cell-free DNA from neurons is greater than about 5%.

9. A method of analyzing a biological sample of a subject, the method comprising:
(i) obtaining cell-free DNA from a blood sample from the subject;
(ii) analyzing a methylation pattern of the cell-free DNA;
(iii) determining a percentage of cell-free DNA from neurons; and
(iv) comparing the percentage of cell-free DNA from neurons to a control.

10. A method of measuring neuron cell death in a subject, the method comprising:
(i) obtaining cell-free DNA from a blood sample from the subject;
(ii) analyzing a methylation pattern of the cell-free DNA;
(iii) determining a percentage of cell-free DNA from neurons; and
(iv) comparing the percentage of cell-free DNA from neurons to a control.

11. A method of selecting a patient for treatment with a therapeutic agent for treatment of a neurodegenerative condition, the method comprising:
(i) obtaining cell-free DNA from a blood sample from the subject;
(ii) analyzing a methylation pattern of the cell-free DNA;
(iii) determining a percentage of cell-free DNA from neurons; and
(iv) comparing the percentage of cell-free DNA from neurons to a control;
wherein the patient is selected for treatment when the percentage of cell-free DNA from neurons is greater than the control.

12. The method of any one of claims 8 to 11, wherein step (ii) comprises analyzing a methylation pattern of whole amplicons of DNA.

13. The method of claim 12, wherein the whole amplicons are at least about 50 base pairs (bp) in length.

14. The method of claim 13, wherein the whole amplicons are between about 50 and about 500 base pairs in length.

15. The method of any one of claims 10 to 14, wherein the patient is selected for treatment when the percentage of cell-free DNA from neurons is greater than about 5%.

16. The method of claim 15, wherein the patient is selected for treatment when the percentage of cell-free DNA from neurons is greater than about 7%.

17. The method of claim 15, wherein the patient is selected for treatment when the percentage of cell-free DNA from neurons is greater than about 9%.

18. The method of any one of claims 1 to 17, wherein a percentage of cell-free DNA from neurons greater than the control indicates an increased risk of neurodegenerative condition or traumatic brain injury.

19. A computer product comprising a non-transitory computer readable medium storing a plurality of instructions that when executed control a computer system to analyze a biological sample from a subject to determine the risk of neurodegenerative condition in the subject, the biological sample comprising cell-free DNA, the instructions comprising:
(i) identifying a first DNA methylation pattern that occurs in a neuron at a rate above a threshold, wherein the first DNA methylation pattern comprises methylation at one or more methylated regions and optionally comprises no methylation at one or more unmethylated regions;
(ii) analyzing a second DNA methylation pattern of the cell-free DNA; and
(iii) computing a relative abundance of the one or more methylated regions and optionally the one or more unmethylated regions in the cell-free DNA; and
(iv) determining the risk of neurodegenerative condition in the subject by comparing the relative abundance to a control.

20. A method for determining efficacy of a potential treatment of a neurodegenerative condition, the method comprising:
(i) obtaining cell-free DNA from a blood sample from a plurality of subjects, wherein the subjects have been administered the potential treatment;
(ii) analyzing a methylation pattern of the cell-free DNA;
(iii) determining a percentage of cell-free DNA from neurons; and
(iv) comparing the percentage of cell-free DNA from neurons to a control;
wherein the potential treatment is efficacious when the percentage of cell-free DNA from neurons is less than the control.

21. The method of claim 20, wherein steps (i)-(iv) are repeated at least once.

22. The method of claim 21, wherein steps (i)-(iv) are repeated weekly.

23. The method of any one of the above claims, wherein determining a percentage of cell-free DNA from neurons comprises comparing the methylation pattern of the cell-free DNA to a neuronal DNA methylation pattern, wherein the neuronal DNA methylation pattern comprises methylation at one or more methylated regions and optionally comprises no methylation at one or more unmethylated regions.

24. A computer-implemented method of analyzing a biological sample, comprising:
(i) identifying a first DNA methylation pattern that occurs in a neuron at a rate above a threshold, wherein the first DNA methylation pattern comprises methylation at one or more methylated regions and optionally comprises no methylation at one or more unmethylated regions;
(ii) analyzing a second DNA methylation pattern of the cell-free DNA; and
(iii) computing a relative abundance of the one or more methylated regions and optionally the one or more unmethylated regions in the cell-free DNA; and
(iv) determining the risk of neurodegenerative disease in the subject by comparing the relative abundance to a control.

25. The method of any one of claims 20 to 24, wherein step (ii) comprises analyzing a methylation pattern of whole amplicons of DNA.

26. The method of claim 25, wherein the whole amplicons are at least about 50 base pairs (bp) in length.

27. The method of claim 26, wherein the whole amplicons are between about 50 and about 500 base pairs in length.

28. The method of any one of the above claims, wherein the control is a percentage of cell-free DNA from neurons in a blood sample from an untreated subject, a blood sample from the subjects prior to treatment, or a threshold.

29. The method of claim 28, wherein the threshold is a percentage of cell-free DNA from neurons greater than about 5%.

30. The method of claim 28, wherein the threshold is a percentage of cell-free DNA from neurons greater than about 7%.

31. The method of claim 28, wherein the threshold is a percentage of cell-free DNA from neurons greater than about 9%.

32. The method of any one of the above claims, wherein the neurodegenerative disease is selected from Alzheimer's disease, Huntington disease, Parkinson's disease, amyotrophic lateral sclerosis (ALS), ataxia, multiple sclerosis, multiple system atrophy, or concussion.

33. The method of any one of the above claims, wherein the neuron is a motor neuron, a sensory neuron, an interneuron, a dopaminergic neuron, cholinergic neuron, a GABAergic neuron, a glutamatergic neuron, or a cortical neuron.

34. The method of any one of the above claims, wherein the neuron is from the forebrain, midbrain, or hindbrain.

35. The method of any one of the above claims, wherein the neuron is from the frontal lobe, temporal lobe, parietal lobe, occipital lobe, cerebellum, or brain stem.

36. The method of any one of the above claims, wherein analyzing the methylation pattern comprises converting 5-methylcytosine in the cell-free DNA to a different nucleotide.

37. The method of claim 36, wherein converting comprises bisulfite conversion or enzymatic conversion.

38. The method of any one of the above claims, wherein the subject has mild cognitive impairment.

39. The method of any one of the above claims, wherein the subject is over 45 years of age.

40. The method of any one of the above claims, comprising selecting a subject over 45 years of age.

41. The method of claim 40, wherein the subject has no symptoms of the neurodegenerative condition.

42. The method of any one of the above claims, comprising selecting a subject having mild cognitive impairment.

43. A method of treating a subject having a mild traumatic brain injury, comprising:
(i) selecting a subject at risk for mild traumatic brain injury;
(ii) obtaining cell-free DNA from a blood sample from the subject;
(iii) analyzing a methylation pattern of the cell-free DNA;
(iv) determining a percentage of cell-free DNA from neurons; and
(v) comparing the percentage of cell-free DNA from neurons to a control;
(vi) treating the subject for mild traumatic brain injury when the percentage of cell-free DNA from neurons is greater than the control.

44. The method of claim 43, wherein treating the subject comprises administering a therapeutic agent that treats one or more symptoms of mild traumatic brain injury.

45. The method of claim 43 or 44, further comprising repeating steps (ii) to (v) at a timepoint after treatment.

46. The method of claim 45, wherein treatment is discontinued when the percentage of cell-free DNA from neurons at the timepoint is less than or equal to the control.

47. The method of claim 46, wherein treatment is continued when the percentage of cell-free DNA from neurons at the timepoint is greater than the control.

48. A kit comprising a first plurality of oligonucleotides, wherein each oligonucleotide in the first plurality is capable of hybridizing to a region that is preferentially methylated in a neuron cell.

49. The kit of claim 48, further comprising a second plurality of oligonucleotides, wherein each oligonucleotide in the second plurality is capable of hybridizing to a region that is preferentially unmethylated in a neuron cell.

50. The kit of claim 48 or 49 for determining efficacy of a potential treatment of a neurodegenerative disease or condition.

51. The kit of claim 50, wherein the neurodegenerative disease or condition is selected from Alzheimer's disease, Huntington disease, Parkinson's disease, amyotrophic lateral sclerosis (ALS), ataxia, multiple sclerosis, multiple system atrophy, or concussion.

52. The kit of any one of claims 48 to 51, wherein the neuron is a motor neuron, a sensory neuron, an interneuron, a dopaminergic neuron, cholinergic neuron, a GABAergic neuron, a glutamatergic neuron, or a cortical neuron.

53. The kit of any one of claims 48 to 52, wherein the neuron is from the forebrain, midbrain, or hindbrain.

54. The kit of any one of claims 48 to 53, wherein the neuron is from the frontal lobe, temporal lobe, parietal lobe, occipital lobe, cerebellum, or brain stem.

55. The kit of any one of claims 48 to 54, wherein the plurality of oligonucleotides comprises one or more oligonucleotides having a nucleotide sequence of any one of SEQ ID NOs: 3-6.

56. A method of detecting cell-free DNA from neurons in a blood sample, comprising:
(i) obtaining cell-free DNA from a blood sample from a human subject; and
(ii) detecting whether cell-free DNA from neurons is present in the blood sample by methylation analysis comprising subjecting the cell-free DNA from the blood sample to sequencing of whole amplicons of DNA.

57. The method of claim 56, wherein the whole amplicons are at least about 50 base pairs (bp) in length.

58. The method of claim 57, wherein the whole amplicons are between about 50 and about 500 base pairs in length.

59. The method of any one of claims 56 to 58, wherein the whole amplicons were produced using one or more primers that comprise the sequence of any one of SEQ ID NOs: 3-6.

60. The method of any one of claims 56 to 59, wherein the whole amplicons were produced using one or more primers targeting a region selected from the regions listed in Table 1.

61. A method for determining the methylation status of an amplicon, comprising:
(i) obtaining cell-free DNA from a blood sample from a human subject;
(ii) converting 5-methylcytosine in the cell-free DNA to a different nucleotide, thereby producing converted cell-free DNA;
(iii) amplifying the converted cell-free DNA, thereby producing an amplicon; and
(iv) sequencing the amplicon, wherein the amplicon is between about 50 bp and about 500 bp in length.

62. The method of claim 61, wherein the whole amplicons were produced using one or more primers that comprise the sequence of any one of SEQ ID NOs: 3-6.

63. The method of claim 61, wherein the whole amplicons were produced using one or more primers targeting a region selected from the regions listed in Table 1.
